(19) United States
(12) Patent Application Publication
(10) Pub. No.: US 2008/0050809 A1
(43) Pub. Date: Feb. 28, 2008

(54) Novel human kinases and polynucleotides encoding the same

(76) Inventors: Alejandro Abuin, The Woodlands, TX (US); Gregory Donoho, Indianapolis, IN (US); Carl Johan Friddle, The Woodlands, TX (US); Glenn Friedrich, Houston, TX (US); Brenda Gerhardt, Spring, TX (US); Erin Hilbun, Denton, TX (US); Yi Hu, Spring, TX (US); James Alvin Kieke, Houston, TX (US); Brian Mathur, Nashville, TN (US); Daniel Mathur, Wooster, OH (US); Maricar Miranda, The Woodlands, TX (US); Michael C. Nehls, Stockdorf (DE); Boris Nepomnichy, Dickinson, TX (US); Arthur T. Sands, The Woodlands, TX (US); John Scoville, Pearland, TX (US); C. Alexander Turner, The Woodlands, TX (US); D. Wade Walke, Spring, TX (US); Xiaoming Wang, Itasca, IL (US); Nathaniel L. Wilganowski, Houston, TX (US); Amy Qiongshu Xie, Needham, MA (US); Xuanchuan Yu, Conroe, TX (US); Brian Zambrowicz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/165,424

(22) Filed: Jun. 23, 2005

Related U.S. Application Data
(63) Continuation-in-part of application No. 10/766,691, filed on Jan. 28, 2004, now abandoned.
Application No. 10/766,691 is a continuation of application No. 09/671,050, filed on Sep. 27, 2000, now Pat. No. 6,716,616.
Application No. 09/671,050 is a continuation-in-part of application No. 10/762,759, filed on Jan. 22, 2004, now abandoned.
Application No. 10/762,759 is a continuation of application No. 09/707,121, filed on Nov. 6, 2000, now Pat. No. 6,720,173.
Application No. 09/707,121 is a continuation-in-part of application No. 10/936,445, filed on Sep. 8, 2004, now abandoned.
Application No. 10/936,445 is a continuation of application No. 10/446,175, filed on May 27, 2003, now Pat. No. 6,806,073.
Application No. 10/446,175 is a continuation of application No. 09/733,388, filed on Dec. 7, 2000, now Pat. No. 6,602,698.
Application No. 09/733,388 is a continuation-in-part of application No. 10/828,828, filed on Apr. 21, 2004, now abandoned.
Application No. 10/828,828 is a continuation of application No. 09/765,068, filed on Jan. 18, 2001, now Pat. No. 6,746,861.
Application No. 09/765,068 is a continuation-in-part of application No. 09/783,320, filed on Feb. 15, 2001, now abandoned.
Application No. 09/783,320 is a continuation-in-part of application No. 10/984,548, filed on Nov. 8, 2004, now abandoned.
Application No. 10/984,548 is a continuation of application No. 10/217,745, filed on Aug. 12, 2002, now Pat. No. 6,838,275.
Application No. 10/217,745 is a continuation of application No. 09/802,117, filed on Mar. 8, 2001, now Pat. No. 6,444,456.
Application No. 09/802,117 is a continuation-in-part of application No. 11/125,295, filed on May 9, 2005, now abandoned.
Application No. 11/125,295 is a continuation of application No. 10/620,845, filed on Jul. 15, 2003, now Pat. No. 6,908,758.
Application No. 10/620,845 is a continuation of application No. 09/841,683, filed on Apr. 24, 2001, now Pat. No. 6,617,147.
Application No. 09/841,683 is a continuation-in-part of application No. 11/025,671, filed on Dec. 29, 2004.
Application No. 11/025,671 is a division of application No. 10/010,720, filed on Nov. 13, 2001, now Pat. No. 6,858,419.
Application No. 10/010,720 is a continuation-in-part of application No. 09/854,856, filed on May 14, 2001, now Pat. No. 6,541,252.
Application No. 09/854,856 is a continuation-in-part of application No. 11/115,086, filed on Apr. 26, 2005, now abandoned.
Application No. 11/115,086 is a continuation of application No. 10/306,879, filed on Nov. 25, 2002, now Pat. No. 6,902,923.
Application No. 10/306,879 is a continuation of application No. 09/883,134, filed on Jun. 15, 2001, now Pat. No. 6,511,840.
Application No. 09/883,134 is a continuation-in-part of application No. 11/046,668, filed on Jan. 28, 2005, now abandoned.
Application No. 11/046,668 is a continuation of application No. 09/940,921, filed on Aug. 28, 2001, now Pat. No. 6,900,045.
Application No. 09/940,921 is a continuation-in-part of application No. 10/462,887, filed on Jun. 17, 2003, now abandoned.
Application No. 10/462,887 is a division of application No. 10/217,357, filed on Aug. 9, 2002, now Pat. No. 6,610,537.
Application No. 10/217,357 is a continuation of application No. 09/975,326, filed on Oct. 11, 2001, now Pat. No. 6,476,210.
Application No. 09/975,326 is a continuation-in-part of application No. 10/843,129, filed on May 11, 2004, now abandoned.
Application No. 10/843,129 is a division of application No. 10/430,797, filed on May 6, 2003, now Pat. No. 6,773,906.
Application No. 10/430,797 is a continuation of application No. 10/004,542, filed on Oct. 23, 2001, now Pat. No. 6,586,230.
Application No. 10/004,542 is a continuation-in-part of application No. 10/948,842, filed on Sep. 23, 2004, now abandoned.
Application No. 10/948,842 is a division of application No. 10/434,034, filed on May 8, 2003, now Pat. No. 6,815,188.
Application No. 10/434,034 is a continuation of application No. 09/992,481, filed on Nov. 19, 2001, now Pat. No. 6,593,125.
Application No. 09/992,481 is a continuation-in-part of application No. 10/934,072, filed on Sep. 3, 2004, now abandoned.
Application No. 10/934,072 is a continuation of application No. 10/419,279, filed on Apr. 17, 2003, now Pat. No. 6,803,221.
Application No. 10/419,279 is a continuation of application No. 10/014,882, filed on Dec. 11, 2001, now Pat. No. 6,593,126.
Application No. 10/014,882 is a continuation-in-part of application No. 11/114,906, filed on Apr. 26, 2005, now abandoned.
Application No. 11/114,906 is a continuation of application No. 10/413,437, filed on Apr. 11, 2003, now Pat. No. 6,902,924.
Application No. 10/413,437 is a continuation of application No. 10/020,079, filed on Dec. 12, 2001, now Pat. No. 6,579,710.
Application No. 10/020,079 is a continuation-in-part of application No. 10/791,666, filed on Mar. 2, 2004, now abandoned.
Application No. 10/791,666 is a continuation of application No. 10/028,946, filed on Dec. 20, 2001, now Pat. No. 6,734,009.
Application No. 10/028,946 is a continuation-in-part of application No. 11/127,567, filed on May 12, 2005.
Application No. 11/127,567 is a continuation of application No. 10/071,879, filed on Feb. 8, 2002, now abandoned.
Application No. 10/071,879 is a continuation-in-part of application No. 10/919,196, filed on Aug. 16, 2004, now abandoned.
Application No. 10/919,196 is a continuation of application No. 10/103,546, filed on Mar. 20, 2002, now abandoned.
Application No. 10/103,546 is a continuation-in-part of application No. 10/655,490, filed on Sep. 4, 2003, now abandoned.
Application No. 10/655,490 is a continuation of application No. 10/103,547, filed on Mar. 20, 2002, now Pat. No. 6,759,527.
Application No. 10/103,547 is a continuation-in-part of application No. 10/803,277, filed on Mar. 18, 2004, now Pat. No. 6,919,192.
Application No. 10/803,277 is a division of application No. 10/116,326, filed on Apr. 4, 2002, now Pat. No. 6,777,545.
Application No. 10/116,326 is a continuation-in-part of application No. 11/035,027, filed on Jan. 13, 2005, now abandoned.
Application No. 11/035,027 is a division of application No. 10/843,136, filed on May 11, 2004, now Pat. No. 6,861,241.
Application No. 10/843,136 is a continuation of application No. 10/116,332, filed on Apr. 4, 2002, now Pat. No. 6,864,079.
Application No. 10/116,332 is a continuation-in-part of application No. 10/798,773, filed on Mar. 11, 2004, now abandoned.
Application No. 10/798,773 is a division of application No. 10/141,634, filed on May 8, 2002, now Pat. No. 6,734,010.
Application No. 10/141,634 is a continuation-in-part of application No. 11/032,674, filed on Jan. 10, 2005, now abandoned.
Application No. 11/032,674 is a division of application No. 10/803,278, filed on Mar. 18, 2004, now Pat. No. 6,861,240.
Application No. 10/803,278 is a division of application No. 10/196,927, filed on May 20, 2002, now Pat. No. 6,797,510.
Application No. 10/196,927 is a continuation-in-part of application No. 10/872,762, filed on Jun. 21, 2004, now abandoned.
Application No. 10/872,762 is a continuation of application No. 10/171,374, filed on Jun. 13, 2002, now Pat. No. 6,841,377.
(60) Provisional application No. 60/156,511, filed on Sep. 28, 1999.
(60) Provisional application No. 60/164,289, filed on Nov. 8, 1999.
(60) Provisional application No. 60/169,428, filed on Dec. 7, 1999.
(60) Provisional application No. 60/176,690, filed on Jan. 18, 2000.
(60) Provisional application No. 60/183,582, filed on Feb. 18, 2000.
(60) Provisional application No. 60/184,014, filed on Feb. 22, 2000.
(60) Provisional application No. 60/188,449, filed on Mar. 10, 2000.
(60) Provisional application No. 60/199,499, filed on Apr. 25, 2000.
(60) Provisional application No. 60/201,227, filed on May 1, 2000.
(60) Provisional application No. 60/206,015, filed on May 19, 2000.
(60) Provisional application No. 60/211,572, filed on Jun. 15, 2000.
(60) Provisional application No. 60/216,382, filed on Jul. 7, 2000.
(60) Provisional application No. 60/229,280, filed on Aug. 31, 2000.
(60) Provisional application No. 60/239,821, filed on Oct. 12, 2000.
(60) Provisional application No. 60/243,893, filed on Oct. 27, 2000.
(60) Provisional application No. 60/252,011, filed on Nov. 20, 2000.
(60) Provisional application No. 60/254,744, filed on Dec. 11, 2000.
(60) Provisional application No. 60/255,103, filed on Dec. 12, 2000.
(60) Provisional application No. 60/289,422, filed on May 8, 2001.
(60) Provisional application No. 60/258,335, filed on Dec. 27, 2000.
(60) Provisional application No. 60/267,583, filed on Feb. 9, 2001.
(60) Provisional application No. 60/278,202, filed on Mar. 23, 2001.
(60) Provisional application No. 60/277,168, filed on Mar. 20, 2001.
(60) Provisional application No. 60/282,036, filed on Apr. 6, 2001.
(60) Provisional application No. 60/282,031, filed on Apr. 6, 2001.
(60) Provisional application No. 60/289,727, filed on May 9, 2001.
(60) Provisional application No. 60/293,248, filed on May 24, 2001.
(60) Provisional application No. 60/297,856, filed on Jun. 13, 2001.

(51) Int. Cl. C12N 5/06; C07H 21/04; C12N 15/00

(52) U.S. Cl. 435/325; 435/320.1; 536/23.2

(57) ABSTRACT

Novel human polynucleotide and polypeptide sequences are disclosed that can be used in therapeutic, diagnostic, and pharmacogenomic applications.

### 1.0 CROSS-REFERENCE TO RELATED APPLICATIONS

The present application is a continuation-in-part of: co-pending U.S. application Ser. No. 10/766,691, filed on Jan. 28, 2004, which is a continuation of U.S. application Ser. No. 09/671,050, filed on Sep. 27, 2000, which issued as U.S. Pat. No. 6,716,616 B1 on Apr. 6, 2004, which claims the benefit of U.S. Provisional Application No. 60/156,511, filed on Sep. 28, 1999; co-pending U.S. application Ser. No. 10/762,759, filed on Jan. 22, 2004, which is a continuation of U.S. application Ser. No. 09/707,121, filed on Nov. 6, 2000, which issued as U.S. Pat. No. 6,720,173 B1 on Apr. 13, 2004, which claims the benefit of U.S. Provisional Application No. 60/164,289, filed on Nov. 8, 1999; co-pending U.S. application Ser. No. 10/936,445, filed on Sep. 8, 2004, which is a continuation of U.S. application Ser. No. 10/446,175, filed on May 27, 2003, which issued as U.S. Pat. No. 6,806,073 B2 on Oct. 19, 2004, which is a continuation of U.S. application Ser. No. 09/733,388, filed on Dec. 7, 2000, which issued as U.S. Pat. No. 6,602,698 B2 on Aug. 5, 2003, which claims the benefit of U.S. Provisional Application No. 60/169,428, filed on Dec. 7, 1999; co-pending U.S. application Ser. No. 10/828,828, filed on Apr. 21, 2004, which is a continuation of U.S. application Ser. No. 09/765,068, filed on Jan. 18, 2001, which issued as U.S. Pat. No. 6,746,861 B2 on Jun. 8, 2004, which claims the benefit of U.S. Provisional Application No. 60/176,690, filed on Jan. 18, 2000; co-pending U.S. application Ser. No. 09/783,320, filed on Feb. 15, 2001, which claims the benefit of U.S. Provisional Application Nos. 60/183,582, filed on Feb. 18, 2000, and 60/184,014, filed on Feb. 22, 2000; co-pending U.S. application Ser. No. 10/984,548, filed on Nov. 8, 2004, which is a continuation of U.S. application Ser. No. 10/217,745, filed on Aug. 12, 2002, which issued as U.S. Pat. No. 6,838,275 B2 on Jan. 4, 2005, which is a continuation of U.S. application Ser. No. 09/802,117, filed on Mar. 8, 2001, which issued as U.S. Pat. No. 6,444,456 B1 on Sep. 3, 2002, which claims the benefit of U.S. Provisional Application No. 60/188,449, filed on Mar. 10, 2000; co-pending U.S. application Ser. No. 11/125,295, filed on May 9, 2005, which is a continuation of co-pending U.S. application Ser. No. 10/620,845, filed on Jul. 15, 2003, which issued as U.S. Pat. No. 6,908,758 B2 on Jun. 21, 2005, which is a continuation of U.S. application Ser. No. 09/841,683, filed on Apr. 24, 2001, which issued as U.S. Pat. No. 6,617,147 B2 on Sep. 9, 2003, which claims the benefit of U.S. Provisional Application Nos. 60/199,499, filed on Apr. 25, 2000, and 60/201,227, filed on May 1, 2000; co-pending U.S. application Ser. No. 11/025,671, filed on Dec. 29, 2004, which is a divisional of U.S. application Ser. No. 10/010,720, filed on Nov. 13, 2001, which issued as U.S. Pat. No. 6,858,419 B1 on Feb. 22, 2005, which is a continuation-in-part of U.S. application Ser. No. 09/854,856, filed on May 14, 2001, which issued as U.S. Pat. No. 6,541,252 B1 on Apr. 1, 2003, which claims the benefit of U.S. Provisional Application No. 60/206,015, filed on May 19, 2000; co-pending U.S. application Ser. No. 11/115,086, filed on Apr. 26, 2005, which is a continuation of U.S. application No. 10/306,879, filed on Nov. 25, 2002, which issued as U.S. Pat. No. 6,902,923 B2 on Jun. 7, 2005, which is a continuation of U.S. application Ser. No. 09/883,134, filed on Jun. 15, 2001, which issued as U.S. Pat. No. 6,511,840 B1 on Jan. 28, 2003, which claims the benefit of U.S. Provisional Application Nos. 60/211,572, filed on Jun. 15, 2000, and 60/216,382, filed on Jul. 7, 2000; co-pending U.S. application Ser. No. 11/046,668, filed on Jan. 28, 2005, which is a continuation of U.S. application Ser. No. 09/940,921, filed on Aug. 28, 2001, which issued as U.S. Pat. No. 6,900,045 B2 on May 31, 2005, which claims the benefit of U.S. Provisional Application No. 60/229,280, filed on Aug. 31, 2000; co-pending U.S. application Ser. No. 10/462,887, filed on Jun. 17, 2003, which is a divisional of U.S. application Ser. No. 10/217,357, filed on Aug. 9, 2002, which issued as U.S. Pat. No. 6,610,537 B2 on Aug. 26, 2003, which is a continuation of U.S. application Ser. No. 09/975,326, filed on Oct. 11, 2001, which issued as U.S. Pat. No. 6,476,210 B2 on Nov. 5, 2002, which claims the benefit of U.S. Provisional Application No. 60/239,821, filed on Oct. 12, 2000; co-pending U.S. application Ser. No. 10/843,129, filed on May 11, 2004, which is a divisional of U.S. application Ser. No. 10/430,797, filed on May 6, 2003, which issued as U.S. Pat. No. 6,773,906 B2 on Aug. 10, 2004, which is a continuation of U.S. application Ser. No. 10/004,542, filed on Oct. 23, 2001, which issued as U.S. Pat. No. 6,586,230 B1 on Jul. 1, 2003, which claims the benefit of U.S. Provisional Application No. 60/243,893, filed on Oct. 27, 2000; co-pending U.S. application Ser. No. 10/948,842, filed on Sep. 23, 2004, which is a divisional of U.S. application Ser. No. 10/434,034, filed on May 8, 2003, which issued as U.S. Pat. No. 6,815,188 B2 on Nov. 9, 2004, which is a continuation of U.S. application Ser. No. 09/992,481, filed on Nov. 19, 2001, which issued as U.S. Pat. No. 6,593,125 B2 on Jul. 15, 2003, which claims the benefit of U.S. Provisional Application No. 60/252,011, filed on Nov. 20, 2000; co-pending U.S. application Ser. No. 10/934,072, filed on Sep. 3, 2004, which is a continuation of U.S. application Ser. No. 10/419,279, filed on Apr. 17, 2003, which issued as U.S. Pat. No. 6,803,221 B2 on Oct. 12, 2004, which is a continuation of U.S. application Ser. No. 10/014,882, filed on Dec. 11, 2001, which issued as U.S. Pat. No. 6,593,126 B2 on Jul. 15, 2003, which claims the benefit of U.S. Provisional Application No. 60/254,744, filed on Dec. 11, 2000; co-pending U.S. application Ser. No. 11/114,906, filed on Apr. 26, 2005, which is a continuation of U.S. application Ser. No. 10/413,437, filed on Apr. 11, 2003, which issued as U.S. Pat. No. 6,902,924 B2 on Jun. 7, 2005, which is a continuation of U.S. application Ser. No. 10/020,079, filed on Dec. 12, 2001, which issued as U.S. Pat. No. 6,579,710 B2 on Jun. 17, 2003, which claims the benefit of U.S. Provisional Application Nos. 60/255,103, filed on Dec. 12, 2000, and 60/289,422, filed on May 8, 2001; co-pending U.S. application Ser. No. 10/791,666, filed on Mar. 2, 2004, which is a continuation of U.S. application Ser. No. 10/028,946, filed on Dec. 20, 2001, which issued as U.S. Pat. No. 6,734,009 B2 on May 11, 2004, which claims the benefit of U.S. Provisional Application No. 60/258,335, filed on Dec. 27, 2000; co-pending U.S. application Ser. No. 11/127,567, filed on May 12, 2005, which is a continuation of U.S. application Ser. No. 10/071,879, filed on Feb. 8, 2002, abandoned, which claims the benefit of U.S. Provisional Application No. 60/267,583, filed on Feb. 9, 2001; co-pending U.S. application Ser. No. 10/919,196, filed on Aug. 16, 2004, which is a continuation of U.S. application Ser. No. 10/103,546, filed on Mar. 20, 2002, abandoned, which claims the benefit of U.S. Provisional Application No. 60/278,202, filed on Mar. 23, 2001; co-pending U.S. application Ser. No. 10/655,490, filed on Sep. 4, 2003, which is a continuation of U.S. application Ser. No. 10/103,547, filed on Mar. 20, 2002, which issued as U.S. Pat. No. 6,759,527 B2 on Jul. 6, 2004, which claims the benefit of U.S. Provisional application Ser. No. 60/277,168, filed on Mar. 20, 2001; co-pending U.S. application Ser. No. 10/803,277, filed on Mar. 18, 2004, which is a divisional of U.S. application Ser. No. 10/116,326, filed on Apr. 4, 2002, which issued as U.S. Pat. No. 6,777,545 B2 on Aug. 17, 2004, which claims the benefit of U.S. Provisional Application No. 60/282,036, filed on Apr. 6, 2001; co-pending U.S. application Ser. No. 11/035,027, filed on Jan. 13, 2005, which is a divisional of U.S. application Ser. No. 10/843,136, filed on May 11, 2004, which issued as U.S. Pat. No. 6,861,241 B2 on Mar. 1, 2005, which is a continuation of U.S. application Ser. No. 10/116,332, filed on Apr. 4, 2002, which issued as U.S. Pat. No. 6,864,079 B2 on Mar. 8, 2005, which claims the benefit of U.S. Provisional Application No. 60/282,031, filed on Apr. 6, 2001; co-pending U.S. application Ser. No. 10/798,773, filed on Mar. 11, 2004, which is a divisional of U.S. application Ser. No. 10/141,634, filed on May 8, 2002, which issued as U.S. Pat. No. 6,734,010 B2 on May 11, 2004, which claims the benefit of U.S. Provisional Application No. 60/289,727, filed on May 9, 2001; co-pending U.S. application Ser. No. 11/032,674, filed on Jan. 10, 2005, which is a divisional of U.S. application Ser. No. 10/803,278, filed on Mar. 18, 2004, which issued as U.S. Pat. No. 6,861,240 B2 on Mar. 1, 2005, which is a divisional of U.S. application Ser. No. 10/196,927, filed on May 20, 2002, which issued as U.S. Pat. No. 6,797,510 B1 on Sep. 28, 2004, which claims the benefit of U.S. Provisional Application No. 60/293,248, filed on May 24, 2001; and co-pending U.S. application Ser. No. 10/872,762, filed on Jun. 21, 2004, which is a continuation of U.S. application Ser. No. 10/171,374, filed on Jun. 13, 2002, which issued as U.S. Pat. No. 6,841,377 B1 on Jan. 11, 2005, which claims the benefit of U.S. Provisional application Ser. No. 60/297,856, filed on Jun. 13, 2001; each of which is herein incorporated by reference in its entirety.

### 2.0 CROSS-REFERENCE TO SEQUENCE LISTING SUBMITTED ON COMPACT DISC

The present application contains a Sequence Listing of SEQ ID NOS:1-279, in file “SEQLIST.txt” (2,891,776 bytes), created on Jun. 23, 2005, submitted herewith on duplicate compact disc (Copy 1 and Copy 2), which is herein incorporated by reference in its entirety.

### 3.0 INTRODUCTION

The present invention relates to the discovery, identification, and characterization of novel human polynucleotides encoding proteins sharing sequence similarity with animal kinases and membrane proteins. The invention encompasses the described polynucleotides, host cell expression systems, the encoded proteins, fusion proteins, polypeptides and peptides, antibodies to the encoded proteins and peptides, and genetically engineered animals that either lack or overexpress the disclosed polynucleotides, antagonists and agonists of the proteins, and other compounds that modulate the expression or activity of the proteins encoded by the disclosed polynucleotides, which can be used for diagnosis, drug screening, clinical trial monitoring, the treatment of diseases and disorders, and cosmetic or nutriceutical applications.

### 4.0 BACKGROUND OF THE INVENTION

Kinases mediate the phosphorylation of a wide variety of proteins and compounds in the cell. Along with phosphatases, kinases are involved in a range of regulatory pathways. Given the physiological importance of kinases, they have been subject to intense scrutiny and are proven drug targets.

Membrane proteins can serve as recognition markers, mediate signal transduction, and can mediate or facilitate the passage of materials across the lipid bilayer. As such, membrane proteins are also proven drug targets.

### 5.0 SUMMARY OF THE INVENTION

The present invention relates to the discovery, identification, and characterization of nucleotides that encode novel human proteins, and the corresponding amino acid sequences of these proteins. The novel human proteins (NHPs) described for the first time herein share structural similarity with animal kinases and membrane proteins, and more particularly: serine/threonine protein kinases, cell division protein kinases, and cyclin dependent kinase (SEQ ID NOS:1-15); calcium/calmodulin-dependent protein kinases and serine/threonine protein kinases (SEQ ID NOS:16-20); serine/threonine protein kinases, casein kinases, and particularly casein kinase I gamma isoforms (SEQ ID NOS:21-23); cell division control protein kinases, serine/threonine protein kinases, NEK2 and NY-REN-55 (SEQ ID NOS:24-29 and 73); membrane-associated guanylate kinases (MAGUKS) (SEQ ID NOS:30-72); G-protein coupled receptor kinases (GRKS) (SEQ ID NOS:74-78); multifunctional calcium/calmodulin dependent protein kinases (SEQ ID NOS:79-81); serine/threonine protein kinases, ribosomal protein kinases, and cAMP-dependant kinases (SEQ ID NOS:82-90); mitogen activated protein (MAP) kinases, serine/threonine protein kinases, P21-activated protein kinases, and NPK1-related protein kinases (SEQ ID NOS:91-154); myosin kinases, particularly myosin III kinases, unconventional myosin classes of proteins, and kinases associated with signal transduction (SEQ ID NOS:155-159); serine/threonine kinases, calcium/calmodulin-dependent kinases, MAP kinases, and kinases associated with signal transduction (SEQ ID NOS:160-165); NIMA (never in mitosis A) related kinases, serine/threonine kinases, calcium/calmodulin-dependent kinases, myosin light chain kinases, and kinases associated with signal transduction (SEQ ID NOS:166-175); serine/threonine kinases, calcium/calmodulin-dependent protein kinases, mitogen activated kinases, and kinases associated with signal transduction (SEQ ID NOS:176-179); receptor tyrosine kinases, particularly ephrin-receptor family kinases, and kinases associated with signal transduction (SEQ ID NOS:180-184); receptor tyrosine kinases, particularly NEK family kinases, serine/threonine kinases, and kinases associated with signal transduction (SEQ ID NOS:185-186); receptor tyrosine kinases, particularly calcium and calmodulin dependent kinases, sequences encoding PK 80, serine/threonine kinases and kinases associated with signal transduction (SEQ ID NOS:187-189); serine/threonine kinases, tyrosine kinases, TGF-beta activated kinases, and a variety of growth factor receptors (SEQ ID NOS:190-192); serine/threonine kinases, casein kinases, calcium/calmodulin-dependent protein kinases, mitogen activated kinases, and kinases associated with signal transduction (SEQ ID NOS:193-232); serine/threonine kinases, Citron kinases, and particularly Citron rho-interacting kinases (full length versions of previously reported proteins that were erroneously presumed to be full length) (SEQ ID NOS:233-236); mammalian proteins having structural domains in common with proteins of the immunoglobulin (Ig) super family, which are often found on the cell surface and can be exploited by human pathogens to gain entry to the cell (SEQ ID NOS:237-247); receptor tyrosine kinases and kinases associated with signal transduction (SEQ ID NO:248-257); receptor tyrosine kinases and especially kinases of the membrane-associated guanylate kinase (MAGUK) family (SEQ ID NOS:258 and 259); serine/threonine kinases, carbon catabolite depressing kinases, and kinases associated with signal transduction (SEQ ID NOS:260-265); serine/threonine kinases, calcium/calmodulin dependent kinases, and myosin light chain kinases (SEQ ID NOS:266 and 267); adenylate kinases, phosphotransferases, and kinases associated with signal transduction (SEQ ID NOS:268-271); serine/threonine kinases, G2 protein kinases, and kinases associated with signal transduction (SEQ ID NOS:272-276); and serine/threonine kinases, calcium/calmodulin dependent kinases, and rho-associated kinases (SEQ ID NOS:277-279). Accordingly, the described NHPs encode novel kinases having homologues and orthologs across a wide range of phyla and species.

The novel human polynucleotides described herein encode open reading frames (ORFs) encoding proteins of: 187, 356, 324, 198, 347, 315, 893, 385, 357, 422, 1035, 1214, 1007, 296, 72, 318, 94, 108, 375, 137, 473, 249, 155, 184, 520, 296, 195, 224, 560, 336, 211, 240, 576, 352, 553, 353, 514, 225, 236, 407, 396, 2382, 2245, 982, 2229, 2092, 829, 2136, 1999, 2354, 2217, 954, 2201, 2064, 801, 2108, 1971, 2322, 2185, 922, 2169, 2032, 769, 2076, 1939, 2294, 2157, 894, 2141, 2004, 741, 2048, 1911, 238, 1236, 974, 922, 255, 683, 654, 388, 398, 766, 765, 942, 308, 692, 817, 1036, 870, 864, 764, 751, 654, 648, 548, 535, 895, 889, 789, 776, 982, 976, 876, 863, 957, 951, 851, 838, 2054, 1958, 311, 223, 606, 497, 635, 2271, 999, 2267, 2042, 1651, 455, 778, 762, 703, 385, 479, 94, 645, 482, and 359 amino acids in length (SEQ ID NOS:2, 4, 6, 8, 10, 12, 15, 17, 19, 22, 25, 27, 29, 31, 33, 35, 37, 39, 41, 43, 45, 47, 49, 51, 53, 55, 57, 59, 61, 63, 65, 67, 69, 71, 75, 77, 80, 83, 85, 87, 89, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 161, 163, 165, 167, 169, 172, 174, 177, 179, 181, 183, 186, 188, 191, 194, 196, 198, 200, 202, 204, 206, 208, 210, 212, 214, 216, 218, 220, 222, 224, 226, 228, 230, 232, 234, 236, 238, 240, 242, 244, 246, 249, 251, 253, 255, 257, 259, 261, 263, 265, 267, 269, 271, 273, 275, and 278, respectively).

The invention also encompasses agonists and antagonists of the described NHPS, including small molecules, large molecules, mutant NHPS, or portions thereof, that compete with native NHPs, peptides, and antibodies, as well as nucleotide sequences that can be used to inhibit the expression of the described NHPs (e.g., antisense and ribozyme molecules, and open reading frame or regulatory sequence replacement constructs) or to enhance the expression of the described NHPs (e.g., expression constructs that place the described polynucleotide under the control of a strong promoter system), and transgenic animals that express a NHP sequence, or “knock-outs” (which can be conditional) that do not express a functional NHP. Knock-out mice can be produced in several ways, one of which involves the use of mouse embryonic stem cell (“ES cell”) lines that contain gene trap mutations in a murine homolog of at least one of the described NHPs. When the unique NHP nucleic acid sequences described in SEQ ID NOS:1-279 are “knocked-out” they provide a method of identifying phenotypic expression of the particular gene, as well as a method of assigning function to previously unknown genes. In addition, animals in which the unique NHP nucleic acid sequences described in SEQ ID NOS:1-279 are “knocked-out” provide a unique source in which to elicit antibodies to homologous and orthologous proteins, which would have been previously viewed by the immune system as “self” and therefore would have failed to elicit significant antibody responses.

Additionally, the unique NHP nucleic acid sequences described in SEQ ID NOS:1-279 are useful for the identification of protein coding sequences, and mapping a unique gene to a particular chromosome. These sequences identify biologically verified exon splice junctions, as opposed to splice junctions that may have been bioinformatically predicted from genomic sequence alone. The sequences of the present invention are also useful as additional DNA markers for restriction fragment length polymorphism (RFLP) analysis, and in forensic biology, particularly given the presence of nucleotide polymorphisms within the described sequences.

Further, the present invention also relates to processes for identifying compounds that modulate, i.e., act as agonists or antagonists of, NHP expression and/or NHP activity that utilize purified preparations of the described NHPs and/or NHP products, or cells expressing the same. Such compounds can be used as therapeutic agents for the treatment of any of a wide variety of symptoms associated with biological disorders or imbalances.

### 6.0 BRIEF DESCRIPTION OF THE FIGURES

No Figures are required in the present invention.

### 7.0 DETAILED DESCRIPTION OF THE INVENTION

The NHPs described for the first time herein are novel proteins that are expressed in, inter alia, human cell lines, and: human brain, pituitary, spinal cord, spleen, trachea, kidney, prostate, testis, and adrenal gland cells (SEQ ID NOS:1-13); human brain, pituitary, cerebellum, spinal cord, thymus, lymph node, bone marrow, trachea, kidney, liver, prostate, testis, thyroid, adrenal gland, pancreas, stomach, small intestine, colon, skeletal muscle, uterus, placenta, mammary gland, adipose, esophagus, bladder, cervix, rectum, pericardium, hypothalamus, ovary, fetal kidney, and fetal lung cells (SEQ ID NOS:14-15); human fetal brain, pituitary, cerebellum, thymus, spleen, trachea, lung, testis, thyroid, adrenal gland, pancreas, colon, uterus, and fetal kidney cells (SEQ ID NOS:16-20); human brain, pituitary, cerebellum, spinal cord, thymus, lymph node, bone marrow, trachea, kidney, liver, prostate, testis, thyroid, adrenal gland, pancreas, salivary gland, stomach, small intestine, colon, skeletal muscle, heart, uterus, placenta, mammary gland, adipose, esophagus, bladder, cervix, rectum, pericardium, hypothalamus, ovary, fetal kidney, and fetal lung cells (SEQ ID NOS:21-23); human fetal brain, brain, pituitary, cerebellum, thymus, spleen, lymph node, bone marrow, trachea, kidney, liver, fetal liver, prostate, testis, thyroid, adrenal gland, pancreas, salivary gland, stomach, small intestine, colon, uterus, placenta, mammary gland, adipose, esophagus, bladder, cervix, rectum, pericardium, hypothalamus, ovary, fetal kidney, and fetal lung cells (SEQ ID NOS:24-29 and 73); human brain, pituitary, cerebellum, thymus, spleen, lymph node, bone marrow, trachea, kidney, liver, fetal liver, prostate, testis, adrenal gland, pancreas, salivary gland, stomach, small intestine, colon, skeletal muscle, uterus, placenta, mammary gland, adipose, skin, esophagus, bladder, rectum, thyroid, umbilical vein endothelial, and fetal lung cells (SEQ ID NOS:30-72); human fetal brain, adult brain, pituitary, cerebellum, spinal cord, thymus, kidney, fetal liver, prostate, testis, adrenal gland, small intestine, skeletal muscle, uterus, placenta, mammary gland, and pericardium cells (SEQ ID NOS:74-78); human brain, pituitary, cerebellum, kidney, prostate, testis, thyroid, adrenal gland, pancreas, salivary gland, heart, uterus, cervix, pericardium, fetal kidney and fetal lung cells (SEQ ID NOS:79-81); human fetal brain, brain, pituitary, cerebellum, spinal cord, trachea, kidney, prostate, testis, thyroid, adrenal gland, pancreas, salivary gland, stomach, adipose, and hypothalamus cells (SEQ ID NOS:82-90); human fetal brain, brain, pituitary, cerebellum, spinal cord, thymus, spleen, lymph node, bone marrow, trachea, kidney, prostate, testis, thyroid, adrenal gland, pancreas, salivary gland, stomach, small intestine, colon, skeletal muscle, uterus, placenta, mammary gland, adipose, esophagus, bladder, cervix, rectum, pericardium, hypothalamus, ovary, fetal kidney, and fetal lung cells (SEQ ID NOS:91-154); human pituitary, lymph node, kidney, testis, thyroid, and fetal kidney cells (SEQ ID NOS:155-159); human pituitary, lymph node, kidney, colon, and prostate cells (SEQ ID NOS:160-165); human fetal brain, brain, pituitary, cerebellum, spinal cord, thymus, spleen, lymph node, bone marrow, trachea, lung, kidney, fetal liver, liver, prostate, testis, thyroid, small intestine, heart, uterus, placenta, mammary gland, adipose, esophagus, cervix, rectum, fetal kidney, and fetal lung cells (SEQ ID NOS:166-170), human pituitary, kidney, thyroid, skeletal muscle, and heart cells (SEQ ID NOS:171-175); human fetal brain, brain, pituitary, spinal cord, testis, adipose, and esophagus cells (SEQ ID NOS:176-179); human fetal brain, brain, pituitary, spinal cord, cerebellum, trachea, kidney, fetal liver, liver, prostate, testis, thyroid, stomach, small intestine, colon, uterus, adipose, esophagus, bladder, cervix, and pericardium cells (SEQ ID NOS:180-184); human pituitary, thymus, spleen, lymph node, bone marrow, trachea, kidney, prostate, testis, thyroid, adrenal gland, pancreas, salivary gland, stomach, small intestine, skeletal muscle, heart, uterus, placenta, adipose, skin, bladder, rectum, pericardium, ovary, fetal kidney, fetal lung, gall bladder, tongue, aorta, 6-, 9-, and 12-week embryo, adenocarcinoma, osteosarcoma, and embryonic carcinoma cells (SEQ ID NOS:185-186); human fetal brain, brain, spinal cord, thymus, lymph node, trachea, lung, prostate, testis, thyroid, adrenal gland, stomach, small intestine, skeletal muscle, uterus, placenta, mammary gland, skin, bladder, pericardium, hypothalamus, fetal kidney, fetal lung, tongue, aorta, 6-, 9-, and 12-week embryo, and embryonic carcinoma cells (SEQ ID NOS:187-189); human fetal brain, brain, pituitary, cerebellum, lymph node, trachea, kidney, liver, prostate, testis, thyroid, adrenal gland, pancreas, stomach, small intestine, colon, skeletal muscle, heart, uterus, and fetal kidney cells (SEQ ID NOS:190-192); human fetal brain, brain, pituitary, cerebellum, fetal lung, kidney, and embryo cells (SEQ ID NOS:193-232); human testis, small intestine, fetal kidney, 6- and 9-week embryo, adenocarcinoma, osteosarcoma, and embryonic carcinoma cells (SEQ ID NOS:233-236); human pituitary, thymus, spleen, lymph node, stomach, mammary gland, fetal kidney, fetal lung, tongue, and 6-week embryo cells (SEQ ID NOS:237-240); human fetal brain, brain, pituitary, cerebellum, spinal cord, thymus, lymph, node, bone marrow, trachea, kidney, fetal liver, prostate, testis, thyroid, adrenal gland, stomach, uterus, placenta, bladder, cervix, hypothalamus, fetal kidney, fetal lung, 6- and 12-week embryo, osteosarcoma, and embryonic carcinoma cells (SEQ ID NOS:241-244); human pituitary, lymph node, ovary and fetal kidney cells (SEQ ID NOS:245-247); human fetal brain, spinal cord, thymus, lymph node, bone marrow, trachea, lung, kidney, fetal liver, liver, prostate, testis, thyroid, pancreas, salivary gland, stomach, small intestine, colon, skeletal muscle, heart, uterus, placenta, mammary gland, adipose, skin, esophagus, bladder, cervix, rectum, fetal kidney, fetal lung, gall bladder, tongue, 6-, 9-, and 12-week old embryo, and osteosarcoma cells (SEQ ID NOS:248-257); human fetal brain, spinal cord, lymph node, bone marrow, adrenal gland, fetal kidney, and fetal lung cells (SEQ ID NOS:258 and 259); human brain, pituitary, cerebellum, spinal cord, lymph node, testis, adrenal gland, uterus, mammary gland, rectum, hypothalamus, ovary, fetal kidney, fetal lung, aorta, 6-, 9-, and 12-week old embryo, adenocarcinoma, osteosarcoma, embryonic carcinoma, and normal umbilical vein cells (SEQ ID NOS:260-265); human fetal brain, lymph node, trachea, kidney, testis, fetal kidney, fetal lung, and 6-week old embryo cells (SEQ ID NOS:266 and 267); human fetal brain, brain, pituitary, cerebellum, spinal cord, thymus, spleen, lymph node, trachea, lung, kidney, fetal liver, prostate, testis, thyroid, adrenal gland, pancreas, salivary gland, stomach, colon, skeletal muscle, heart, uterus, placenta, mammary gland, skin, esophagus, bladder, cervix, rectum, pericardium, hypothalamus, ovary, fetal kidney, fetal lung, gall bladder, tongue, aorta, 6-, 9-, and 12-week old embryo, adenocarcinoma, osteosarcoma, embryonic carcinoma, umbilical vein, microvascular endothelial, bone marrow and adipose cells (SEQ ID NOS:268-271); human fetal brain, brain, lung, pituitary, kidney, lymph node, testis, thyroid, adrenal gland, fetal kidney, fetal lung and osteosarcoma cells (SEQ ID NOS:272-276); and human lymph node, bone marrow, esophagus, fetal kidney, fetal lung, tongue, adenocarcinoma, and osteocarcinoma cells (SEQ ID NOS:277-279).

The described sequences were compiled from: gene trapped cDNAs and sequences isolated from a human testis cDNA library (SEQ ID NOS:1-13); gene trapped cDNAs, ESTS, and sequences isolated from a human brain cDNA library (SEQ ID NOS:14-15); gene trapped cDNAs, ESTs, and sequences isolated from a human thymus cDNA library (SEQ ID NOS:16-20); gene trapped cDNAs, ESTs, and sequences isolated from human prostate and testis cDNA libraries (SEQ ID NOS:21-23); gene trapped sequences in conjunction with sequences available in GenBank (SEQ ID NOS:24-29 and 73); gene trapped sequences in conjunction with sequences available in GenBank, and sequences isolated from lung and testis cDNAs libraries (SEQ ID NOS:30-72); gene trapped sequences in conjunction with sequences available in GenBank, and sequences isolated from adrenal gland, skeletal muscle, thymus, and testis cDNA libraries (SEQ ID NOS:74-78); human genomic sequence and sequences isolated from human trachea and testis cDNA libraries (SEQ ID NOS:79-81); gene trapped sequences in conjunction with sequences available in GenBank, and sequences isolated from testis, brain, and kidney cDNA libraries (SEQ ID NOS:82-90); gene trapped sequences in conjunction with sequences available in GenBank, and sequences isolated from a brain cDNA library (SEQ ID NOS:91-154); gene trapped sequences in conjunction with sequences available in GenBank, and sequences isolated from a kidney cDNA library (SEQ ID NOS:155-159); sequences available in GenBank, and sequences isolated from kidney, prostate, and colon cDNA libraries (SEQ ID NOS:160-165); human gene trapped products, sequences available in GenBank, and sequences isolated from kidney, testis, trachea, esophagus, and pituitary cDNA libraries (SEQ ID NOS:166-170), sequences available in GenBank, and sequences isolated from bone marrow and skeletal muscle cDNA libraries (SEQ ID NOS:171-175); sequences available in GenBank, and sequences isolated from skeletal muscle, adipose, pituitary, cerebellum, and brain cDNA libraries (SEQ ID NOS:176-179); sequences available in GenBank, and sequences isolated from prostate and testis cDNA libraries (SEQ ID NOS:180-184); sequences available in GenBank, and sequences isolated from pituitary, lymph node, mammary gland, brain, adrenal gland, fetus, and testis cDNA libraries (SEQ ID NOS:185-189); human genomic sequence and sequences isolated from human brain, lymph node, liver, cerebellum, kidney, testis, and bone marrow cDNA libraries (SEQ ID NOS:190-192); sequences available in GenBank, and sequences isolated from human brain and cerebellum cDNA libraries identified using primers generated from human genomic DNA (SEQ ID NOS:193-232); sequences available in GenBank (Accession Number AC016922), and sequences isolated from human fetal kidney, testis, and lymph node cDNA libraries identified using primers generated from human genomic DNA (SEQ ID NOS:233-236); clustered genomic sequence, ESTs, and sequences isolated from activated T-cell, thymus, and lymph node cDNA libraries (SEQ ID NOS:237-240); sequences isolated from pituitary, lymph node, germ cell tumor, retinal blastoma, glioblastoma, and papillary carcinoma cDNA libraries (SEQ ID NOS:245-247); sequences available in GenBank, and sequences isolated from a human kidney cDNA library (SEQ ID NOS:248-257); sequences available in GenBank, and sequences isolated from fetal kidney, spleen, and spinal cord cDNA libraries (SEQ ID NOS:258 and 259); sequences available in GenBank, and sequences isolated from human adult and fetal brain, pituitary, hypothalamus, testis, and fetus cDNA libraries identified using primers generated from human genomic DNA (SEQ ID NOS:260-265); human genomic sequence, and sequences isolated from human testis, kidney, lymph node, trachea, and fetal brain cDNA libraries (SEQ ID NOS:266 and 267); sequences available in GenBank, and sequences isolated from a human mammary gland cDNA library identified using primers generated from human genomic DNA (SEQ ID NOS:268-271); sequences available in GenBank, and sequences isolated from human lymph node, brain, fetal brain, thyroid, and testis cDNA libraries identified using primers generated from human genomic DNA (SEQ ID NOS:272-276); and human genomic sequence, and sequences isolated from human fetal lung, fetal kidney, esophagus, and lymph node cDNA libraries (SEQ ID NOS:277-279). The cDNA libraries were purchased from Clontech (Palo Alto, Calif.) and/or Edge Biosystems (Gaithersburg, Md.).

The present invention encompasses the nucleotides presented in the Sequence Listing, host cells expressing such nucleotides, the expression products of such nucleotides, and: (a) nucleotides that encode mammalian homologs of the described nucleotides, including the specifically described NHPs, and the NHP products; (b) nucleotides that encode one or more portions of a NHP that correspond to functional domains, and the polypeptide products specified by such nucleotide sequences, including, but not limited to, the novel regions of any active domain(s); (c) isolated nucleotides that encode mutant versions, engineered or naturally occurring, of the described NHPs, in which all or a part of at least one domain is deleted or altered, and the polypeptide products specified by such nucleotide sequences, including, but not limited to, soluble proteins and peptides; (d) nucleotides that encode chimeric fusion proteins containing all or a portion of a coding region of a NHP, or one of its domains (e.g., a receptor/ligand binding domain, accessory protein/self-association domain, etc.) fused to another peptide or polypeptide; or (e) therapeutic or diagnostic derivatives of the described polynucleotides, such as oligonucleotides, antisense polynucleotides, ribozymes, dsRNA, or gene therapy constructs, comprising a sequence first disclosed in the Sequence Listing.

As discussed above, the present invention includes the human DNA sequences presented in the Sequence Listing (and vectors comprising the same), and additionally contemplates any nucleotide sequence encoding a contiguous NHP open reading frame (ORF) that hybridizes to a complement of a DNA sequence presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1, p. 2.10.3 (Ausubel et al., eds., Green Publishing Associates, Inc., and John Wiley & Sons, Inc., New York, 1989)) and encodes a functionally equivalent expression product. Additionally contemplated are any nucleotide sequences that hybridize to the complement of a DNA sequence that encodes and expresses an amino acid sequence presented in the Sequence Listing under moderately stringent conditions, e.g., washing in 0.2×SSC/0.1% SDS at 42° C. (“Current Protocols in Molecular Biology”, supra), yet still encode a functionally equivalent NHP product. Functional equivalents of a NHP include naturally occurring NHPs present in other species, and mutant NHPs, whether naturally occurring or engineered (by site directed mutagenesis, gene shuffling, or directed evolution). The invention also includes degenerate nucleic acid variants of the disclosed NHP polynucleotide sequences.

Additionally contemplated are polynucleotides encoding NHP ORFs, or their functional equivalents, encoded by polynucleotide sequences that are about 99, 95, 90, or about 85 percent similar to corresponding regions of SEQ ID NOS:1-279 (as measured by BLAST sequence comparison analysis using, for example, the GCG sequence analysis package (the University of Wisconsin GCG sequence analysis package, SEQUENCHER 3.0, Gene Codes Corp., Ann Arbor, Mich.) using default parameters).

The invention also includes nucleic acid molecules, preferably DNA molecules, that hybridize to, and are therefore the complements of, the described NHP-encoding polynucleotides. Such hybridization conditions can be highly stringent or less highly stringent, as described herein. In instances where the nucleic acid molecules are deoxyoligonucleotides (“DNA oligos”), such molecules are generally about 16 to about 100 bases long, or about 20 to about 80 bases long, or about 34 to about 45 bases long, or any variation or combination of sizes represented therein that incorporate a contiguous region of sequence first disclosed in the Sequence Listing. Such oligonucleotides can be used in conjunction with the polymerase chain reaction (PCR) to screen libraries, isolate clones, and prepare cloning and sequencing templates, etc.

Alternatively, such NHP oligonucleotides can be used as hybridization probes for screening libraries, and assessing gene expression patterns (particularly using a microarray or high-throughput “chip” format). Additionally, a series of NHP oligonucleotide sequences, or the complements thereof, can be used to represent all or a portion of the described NHP sequences. An oligonucleotide or polynucleotide sequence first disclosed in at least a portion of one or more of the nucleic acid sequences of SEQ ID NOS:1-279 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the nucleic acid sequences of SEQ ID NOS:1-279, or an amino acid sequence encoded thereby. Methods for attaching biopolymers to, or synthesizing biopolymers on, solid support matrices, and conducting binding studies thereon, are disclosed in, inter alia, U.S. Pat. Nos. 5,700,637, 5,556,752, 5,744,305, 4,631,211, 5,445,934, 5,252,743, 4,713,326, 5,424,186, and 4,689,405.

Addressable arrays comprising sequences first disclosed in SEQ ID NOS:1-279 can be used to identify and characterize the temporal and tissue specific expression of a gene. These addressable arrays incorporate oligonucleotide sequences of sufficient length to confer the required specificity, yet be within the limitations of the production technology. The length of these probes is usually within a range of between about 8 to about 2000 nucleotides. Preferably the probes consist of 60 nucleotides, and more preferably 25 nucleotides, from the nucleic acid sequences first disclosed in SEQ ID NOS:1-279.

For example, a series of NHP oligonucleotide sequences, or the complements thereof, can be used in chip format to represent all or a portion of the described sequences. The oligonucleotides, typically between about 16 to about 40 (or any whole number within the stated range) nucleotides in length, can partially overlap each other, and/or the sequence may be represented using oligonucleotides that do not overlap. Accordingly, the described polynucleotide sequences shall typically comprise at least about two or three distinct oligonucleotide sequences of at least about 8 nucleotides in length that are each first disclosed in the described Sequence Listing. Such oligonucleotide sequences can begin at any nucleotide present within a sequence in the Sequence Listing, and proceed in either a sense (5′-to-3′) orientation vis-a-vis the described sequence or in an antisense (3′-to-5′) orientation.

Microarray-based analysis allows the discovery of broad patterns of genetic activity, providing new understanding of gene functions, and generating novel and unexpected insight into transcriptional processes and biological mechanisms. The use of addressable arrays comprising sequences first disclosed in SEQ ID NOS:1-279 provides detailed information about transcriptional changes involved in a specific pathway, potentially leading to the identification of novel components, or gene functions that manifest themselves as novel phenotypes.

Probes consisting of sequences first disclosed in SEQ ID NOS:1-279 can also be used in the identification, selection, and validation of novel molecular targets for drug discovery. The use of these unique sequences permits the direct confirmation of drug targets, and recognition of drug dependent changes in gene expression that are modulated through pathways distinct from the intended target of the drug. These unique sequences therefore also have utility in defining and monitoring both drug action and toxicity.

As an example of utility, the sequences first disclosed in SEQ ID NOS:1-279 can be utilized in microarrays, or other assay formats, to screen collections of genetic material from patients who have a particular medical condition. These investigations can also be carried out using the sequences first disclosed in SEQ ID NOS:1-279 in silico, and by comparing previously collected genetic databases and the disclosed sequences using computer software known to those in the art. Thus the sequences first disclosed in SEQ ID NOS:1-279 can be used to identify mutations associated with a particular disease, and also in diagnostic or prognostic assays.

Although the presently described sequences have been specifically described using nucleotide sequence, it should be appreciated that each of the sequences can uniquely be described using any of a wide variety of additional structural attributes, or combinations thereof. For example, a given nucleic acid sequence can be described by the net composition of the nucleotides present within a given region of the sequence, in conjunction with the presence of one or more specific oligonucleotide sequence(s) first disclosed in SEQ ID NOS:1-279. Alternatively, a restriction map specifying the relative positions of restriction endonuclease digestion sites, or various palindromic or other specific oligonucleotide sequences, can be used to structurally describe a given sequence. Such restriction maps, which are typically generated by widely available computer programs (e.g., the University of Wisconsin GCG sequence analysis package, SEQUENCHER 3.0, Gene Codes Corp., etc.), can optionally be used in conjunction with one or more discrete nucleotide sequence(s) present in the sequence that can be described by the relative position of the sequence relative to one or more additional sequence(s) or one or more restriction sites present in the disclosed sequence.

For oligonucleotide probes, highly stringent conditions may refer, e.g., to washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides). These nucleic acid molecules may encode or act as NHP antisense molecules, useful, for example, in NHP gene regulation and/or as antisense primers in amplification reactions of NHP nucleic acid sequences. With respect to NHP gene regulation, such techniques can be used to regulate biological functions. Further, such sequences can be used as part of ribozyme and/or triple helix sequences that are also useful for NHP gene regulation.

Inhibitory antisense or double stranded oligonucleotides can additionally comprise at least one modified base moiety that is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine.

The antisense oligonucleotide can also comprise at least one modified sugar moiety selected from the group including, but not limited to, arabinose, 2-fluoroarabinose, xylulose, and hexose.

In yet another embodiment, the antisense oligonucleotide will comprise at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641, 1987). The oligonucleotide is a 2′-O-methylribonucleotide (Inoue et al., Nucl. Acids Res. 15:6131-6148, 1987), or a chimeric RNA-DNA analogue (Inoue et al., FEBS Lett. 215:327-330, 1987). Alternatively, double stranded RNA can be used to disrupt the expression and function of a targeted NHP.

Oligonucleotides of the invention can be synthesized by standard methods known in the art, e.g., by use of an automated DNA synthesizer (such as are commercially available from Biosearch, Applied Biosystems, etc.). As examples, phosphorothioate oligonucleotides can be synthesized by the method of Stein et al. (Nucl. Acids Res. 16:3209-3221, 1988), and methylphosphonate oligonucleotides can be prepared by use of controlled pore glass polymer supports (Sarin et al., Proc. Natl. Acad. Sci. USA 85:7448-7451, 1988), etc.

Low stringency conditions are well-known to those of skill in the art, and will vary predictably depending on the specific organisms from which the library and the labeled sequences are derived. For guidance regarding such conditions, see, for example, “Molecular Cloning, A Laboratory Manual” (Sambrook et al., eds., Cold Spring Harbor Press, Cold Spring Harbor, N.Y., 1989), “Current Protocols in Molecular Biology”, supra, and periodic updates thereof.

Alternatively, suitably labeled NHP nucleotide probes can be used to screen a human genomic library using appropriately stringent conditions or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics and pharmacogenomics.

For example, the present sequences can be used in restriction fragment length polymorphism (RFLP) analysis to identify specific individuals. In this technique, an individual's genomic DNA is digested with one or more restriction enzymes, and probed on a Southern blot to yield unique bands for identification (as generally described in U.S. Pat. No. 5,272,057). In addition, the sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). Actual base sequence information can be used for identification as an accurate alternative to patterns formed by restriction enzyme generated fragments.

Further, a NHP gene homolog can be isolated from nucleic acid from an organism of interest by performing PCR using two degenerate or “wobble” oligonucleotide primer pools designed on the basis of amino acid sequences within the NHP products disclosed herein. The template for the reaction may be total RNA, mRNA, genomic DNA and/or cDNA obtained by reverse transcription of mRNA prepared from, for example, human or non-human cell lines or tissue known to express, or suspected of expressing, an allele of a NHP gene.

The PCR product can be subcloned and sequenced to ensure that the amplified sequences represent the sequence of the desired NHP gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library.

PCR technology can also be used to isolate full length cDNA sequences. For example, RNA can be isolated, following standard procedures, from an appropriate cellular or tissue source (i.e., one known to express, or suspected of expressing, a NHP gene). A reverse transcription (RT) reaction can be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment can be isolated. For a review of cloning strategies that can be used, see, e.g., “Molecular Cloning, A Laboratory Manual”, supra.

A cDNA encoding a mutant NHP sequence can be isolated, for example, by using PCR. In this case, the first cDNA strand may be synthesized by hybridizing an oligo-dT oligonucleotide to mRNA isolated from tissue known to express, or suspected of expressing, a NHP, in an individual putatively carrying a mutant NHP allele, and by extending the new strand with reverse transcriptase. The second strand of the cDNA is then synthesized using an oligonucleotide that hybridizes specifically to the 5′ end of the normal sequence. Using these two primers, the product is then amplified via PCR, optionally cloned into a suitable vector, and subjected to DNA sequence analysis through methods well-known to those of skill in the art. By comparing the DNA sequence of the mutant NHP allele to that of a corresponding normal NHP allele, the mutation(s) responsible for the loss or alteration of function of the mutant NHP gene product can be ascertained.

Alternatively, a genomic library can be constructed using DNA obtained from an individual suspected of carrying, or known to carry, a mutant NHP allele (e.g., a person manifesting a NHP-associated phenotype such as, for example, behavioral disorders, immune disorders, osteoporosis, obesity, high blood pressure, connective tissue disorders, infertility, etc.), or a cDNA library can be constructed using RNA from a tissue known to express, or suspected of expressing, a mutant NHP allele. A normal NHP gene, or any suitable fragment thereof, can then be labeled and used as a probe to identify the corresponding mutant NHP allele in such libraries. Clones containing mutant NHP sequences can then be purified and subjected to sequence analysis according to methods well-known to those skilled in the art.

Additionally, an expression library can be constructed utilizing cDNA synthesized from, for example, RNA isolated from a tissue known to express, or suspected of expressing, a mutant NHP allele in an individual suspected of carrying, or known to carry, such a mutant allele. In this manner, gene products made by the putatively mutant tissue may be expressed and screened using standard antibody screening techniques in conjunction with antibodies raised against a normal NHP product, as described below (for screening techniques, see, for example, “Antibodies: A Laboratory Manual” (Harlow and Lane, eds., Cold Spring Harbor Press, Cold Spring Harbor, N.Y., 1988)).

Additionally, screening can be accomplished by screening with labeled NHP fusion proteins, such as, for example, alkaline phosphatase-NHP or NHP-alkaline phosphatase fusion proteins. In cases where a NHP mutation results in an expression product with altered function (e.g., as a result of a missense or a frameshift mutation), polyclonal antibodies to a NHP are likely to cross-react with a corresponding mutant NHP expression product. Library clones detected via their reaction with such labeled antibodies can be purified and subjected to sequence analysis according to methods well-known in the art.

An additional application of the described novel human polynucleotide sequences is their use in the molecular mutagenesis/evolution of proteins that are at least partially encoded by the described novel sequences using, for example, polynucleotide shuffling or related methodologies. Such approaches are described in U.S. Pat. Nos. 5,830,721, 5,837,458, 6,117,679, and 5,723,323.

The invention also encompasses: (a) DNA vectors that contain any of the foregoing NHP coding sequences and/or their complements (i.e., antisense); (b) DNA expression vectors that contain any of the foregoing NHP coding sequences operatively associated with a regulatory element that directs the expression of the coding sequences (for example, baculovirus as described in U.S. Pat. No. 5,869,336); (c) genetically engineered host cells that contain any of the foregoing NHP coding sequences operatively associated with a regulatory element that directs the expression of the coding sequences in the host cell; and (d) genetically engineered host cells that express an endogenous NHP sequence under the control of an exogenously introduced regulatory element (i.e., gene activation). As used herein, regulatory elements include, but are not limited to, inducible and non-inducible promoters, enhancers, operators, and other elements known to those skilled in the art that drive and regulate expression. Such regulatory elements include, but are not limited to, the cytomegalovirus (hCMV) immediate early gene, regulatable, viral elements (particularly retroviral LTR promoters), the early or late promoters of SV40 or adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

Where, as in the present instance, some of the described NHP peptides or polypeptides are thought to be cytoplasmic or nuclear proteins (although processed forms or fragments can be secreted or membrane associated), expression systems can be engineered that produce soluble derivatives of a NHP (corresponding to a NHP extracellular and/or intracellular domains, or truncated polypeptides lacking one or more hydrophobic domains) and/or NHP fusion protein products (especially NHP-Ig fusion proteins, i.e., fusions of a NHP domain to an IgFc), NHP antibodies, and anti-idiotypic antibodies (including Fab fragments) that can be used in therapeutic applications. Preferably, the above expression systems are engineered to allow the desired peptide or polypeptide to be recovered from the culture media.

The present invention also encompasses antibodies and anti-idiotypic antibodies (including Fab fragments), antagonists and agonists of a NHP, as well as compounds or nucleotide constructs that inhibit expression of a NHP sequence (transcription factor inhibitors, antisense and ribozyme molecules, or open reading frame sequence or regulatory sequence replacement constructs), or promote the expression of a NHP (e.g., expression constructs in which NHP coding sequences are operatively associated with expression control elements such as promoters, promoter/enhancers, etc.).

The NHPs or NHP peptides, NHP fusion proteins, NHP nucleotide sequences, antibodies, antagonists and agonists can be useful for the detection of mutant NHPs, or inappropriately expressed NHPs, for the diagnosis of disease. The NHP proteins or peptides, NHP fusion proteins, NHP nucleotide sequences, host cell expression systems, antibodies, antagonists, agonists and genetically engineered cells and animals can be used for screening for drugs (or high throughput screening of combinatorial libraries) effective in the treatment of the symptomatic or phenotypic manifestations of perturbing the normal function of a NHP in the body. The use of engineered host cells and/or animals can offer an advantage in that such systems allow not only for the identification of compounds that bind to the endogenous receptor/ligand of a NHP, but can also identify compounds that trigger NHP-mediated activities or pathways.

Finally, the NHP products can be used as therapeutics. For example, soluble derivatives such as NHP peptides/domains corresponding to NHPs, NHP fusion protein products (especially NHP-Ig fusion proteins, i.e., fusions of a NHP, or a domain of a NHP, to an IgFc), NHP antibodies and anti-idiotypic antibodies (including Fab fragments), antagonists or agonists (including compounds that modulate or act on downstream targets in a NHP-mediated pathway) can be used to directly treat diseases or disorders. For instance, the administration of an effective amount of a soluble NHP, a NHP-IgFc fusion protein, or an anti-idiotypic antibody (or its Fab) that mimics the NHP, could activate or effectively antagonize the endogenous NHP or a protein interactive therewith. Nucleotide constructs encoding such NHP products can be used to genetically engineer host cells to express such products in vivo; these genetically engineered cells function as “bioreactors” in the body delivering a continuous supply of a NHP, a NHP peptide, or a NHP fusion protein to the body. Nucleotide constructs encoding functional NHPs, mutant NHPs, as well as antisense and ribozyme molecules, can also be used in “gene therapy” approaches for the modulation of NHP expression. Thus, the invention also encompasses pharmaceutical formulations and methods for treating biological disorders.

Various aspects of the invention are described in greater detail in the subsections below.

### 7.1 The NHP Sequences

The cDNA sequences and corresponding deduced amino acid sequences of the described NHPs (SEQ ID NOS:1-279) are presented in the Sequence Listing.

A number of polymorphisms were identified during the sequencing of the disclosed nucleic acid sequences, including: an A/G polymorphism (denoted by “r” in the Sequence Listing) in the 3′ UTR of SEQ ID NO:13 (which includes a complete NHP ORF flanked by 5′ and 3′ sequences); an A/G polymorphism at nucleotide (nt) position 739 of SEQ ID NOS:30, 34, 40, 44, 52, 60 and 68, and nt position 67 of SEQ ID NOS:32, 36, 46, 54, 62 and 70, which can result in an isoleucine or valine residue at corresponding amino acid (aa) position number 247 of SEQ ID NOS:31, 35, 41, 45, 53, 61 and 69, and aa position number 23 of SEQ ID NOS:33, 37, 47, 55, 63 and 71; a silent C/T polymorphism (denoted by “y” in the Sequence Listing) at nt position 24 of SEQ ID NO:74, both of which result in an aspartate residue at corresponding aa position 8 of SEQ ID NO:75; a silent C/G polymorphism at nt position 9 of SEQ ID NOS:82, 84, 86 and 88, both of which result in an alanine residue at corresponding aa position 3 of SEQ ID NOS:83, 85, 87 and 89; an A/T polymorphism (denoted by “w” in the Sequence Listing) at nt position 2929 of SEQ ID NOS:91, 93, and 95, nt position 2470 of SEQ ID NOS:97, 99, and 101, nt position 2845 of SEQ ID NOS:107, 109, and 111, nt position 2386 of SEQ ID NOS:113, 115, and 117, nt position 2749 of SEQ ID NOS:123, 125, and 127, nt position 2290 of SEQ ID NOS:129, 131, and 133, nt position 2665 of SEQ ID NOS:139, 141, and 143, and nt position 2206 of SEQ ID NOS:145, 147, and 149, which can result in a serine or threonine residue at corresponding aa position 977 of SEQ ID NOS:92, 94, and 96, aa position 824 of SEQ ID NOS:98, 100, and 102, aa position 949 of SEQ ID NOS:108, 110, and 112, aa position 796 of SEQ ID NOS:114, 116, and 118, aa position 917 of SEQ ID NOS:124, 126, and 128, aa position 764 of SEQ ID NOS:130, 132, and 134, aa position 889 of SEQ ID NOS:140, 142, and 144, and aa position 736 of SEQ ID NOS:146, 148, and 150, respectively; a G/T polymorphism (denoted by “k” in the Sequence Listing) at nt position 5424 of SEQ ID NOS:91 and 93, nt position 4965 of SEQ ID NOS:97 and 99, nt position 4686 of SEQ ID NOS:103 and 105, nt position 5340 of SEQ ID NOS:107 and 109, nt position 4881 of SEQ ID NOS:113 and 115, nt position 4602 of SEQ ID NOS:119 and 121, nt position 5244 of SEQ ID NOS:123 and 125, nt position 4785 of SEQ ID NOS:129 and 131, nt position 4506 of SEQ ID NOS:135 and 137, nt position 5160 of SEQ ID NOS:139 and 141, nt position 4701 of SEQ ID NOS:145 and 147, and nt position 4422 of SEQ ID NOS:151 and 153, which can result in a methionine or isoleucine residue at corresponding aa position 1808 of SEQ ID NOS:92 and 94, aa position 1655 of SEQ ID NOS:98 and 100, aa position 1562 of SEQ ID NOS:104 and 106, aa position 1780 of SEQ ID NOS:108 and 110, aa position 1627 of SEQ ID NOS:114 and 116, aa position 1534 of SEQ ID NOS:120 and 122, aa position 1748 of SEQ ID NOS:124 and 126, aa position 1595 of SEQ ID NOS:130 and 132, aa position 1502 of SEQ ID NOS:136 and 138, aa position 1720 of SEQ ID NOS:140 and 142, aa position 1567 of SEQ ID NOS:146 and 148, and aa position 1474 of SEQ ID NOS:152 and 154, respectively; a silent T/C polymorphism (denoted by “y” in the Sequence Listing) at nt position 3960 of SEQ ID NOS:91 and 93, nt position 3501 of SEQ ID NOS:97 and 99, nt position 3222 of SEQ ID NOS:103 and 105, nt position 3876 of SEQ ID NOS:107 and 109, nt position 3417 of SEQ ID NOS:113 and 115, nt position 3138 of SEQ ID NOS:119 and 121, nt position 3780 of SEQ ID NOS:123 and 125, nt position 3321 of SEQ ID NOS:129 and 131, nt position 3042 of SEQ ID NOS:135 and 137, nt position 3696 of SEQ ID NOS:139 and 141, nt position 3237 of SEQ ID NOS:145 and 147, and nt position 2958 of SEQ ID NOS:151 and 153, both of which lead to an asparagine residue at corresponding aa position 1320 of SEQ ID NOS:92 and 94, aa position 1167 of SEQ ID NOS:98 and 100, aa position 1074 of SEQ ID NOS:104 and 106, aa position 1292 of SEQ ID NOS:108 and 110, aa position 1139 of SEQ ID NOS:114 and 116, aa position 1046 of SEQ ID NOS:120 and 122, aa position 1260 of SEQ ID NOS:124 and 126, aa position 1107 of SEQ ID NOS:130 and 132, aa position 1014 of SEQ ID NOS:136 and 138, aa position 1232 of SEQ ID NOS:140 and 142, aa position 1079 of SEQ ID NOS:146 and 148, and aa position 986 of SEQ ID NOS:152 and 154, respectively; a silent C/T polymorphism (denoted by “y” in the Sequence Listing) at nt position 309 of SEQ ID NOS:155 and 157, both of which result in a threonine residue at corresponding aa position 103 of SEQ ID NOS:156 and 158; an A/G polymorphism (denoted by “r” in the Sequence Listing) at nt position 889 of SEQ ID NO:157, which can result in a lysine or glutamate residue at corresponding aa position 297 of SEQ ID NO:158; a silent C/T polymorphism (denoted by “y” in the Sequence Listing) at nt position 1188 of SEQ ID NO:157, both of which result in an asparagine residue at corresponding aa position 396 of SEQ ID NO:158; a silent A/G polymorphism (denoted by “r” in the Sequence Listing) at nt position 1317 of SEQ ID NO:157, both of which result in a glutamine residue at corresponding aa position 439 of SEQ ID NO:158; a silent C/T polymorphism (denoted by “y” in the Sequence Listing) at nt position 3121 of SEQ ID NO:157, both of which result in a leucine residue at corresponding aa position 1041 of SEQ ID NO:158; a C/T polymorphism (denoted by “y” in the Sequence Listing) at nt position 1043 of SEQ ID NOS:160 and 162, which can result in a serine or phenylalanine residue at corresponding aa position 348 of SEQ ID NOS:161 and 163; a silent T/C polymorphism (denoted by “y” in the Sequence Listing) at nt position 294 of SEQ ID NOS:176 and 178, both of which result in a serine residue at corresponding aa position 98 of SEQ ID NOS:177 and 179; a silent T/C polymorphism (denoted by “y” in the Sequence Listing) at nt position 1170 of SEQ ID NO:185, both of which result in a glycine residue at corresponding aa position 390 of SEQ ID NO:186; a silent T/C polymorphism (denoted by “y” in the Sequence Listing) at nt position 1321 of SEQ ID NO:185, both of which result a leucine residue at corresponding aa position 441 of SEQ ID NO:186; a C/G polymorphism at nt position 94 of SEQ ID NO:187, which can result in a leucine or valine residue at corresponding aa position 32 of SEQ ID NO:188; an A/G polymorphism at nt position 112 of SEQ ID NO:187, which can result in a lysine or glutamate residue at corresponding aa position 38 of SEQ ID NO:188; an A/T polymorphism at nt position 133 of SEQ ID NO:187, which can result in a threonine or serine residue at corresponding aa position 45 of SEQ ID NO:188; an A/G polymorphism (denoted by “r” in the Sequence Listing) at nt position 2182 of SEQ ID NO:190, which can result in a valine or isoleucine residue at corresponding aa position 728 of SEQ ID NO:191; a G/T polymorphism (denoted by “k” in the Sequence Listing) at nt position 2223 of SEQ ID NO:190, which can result in a glutamate or aspartate residue at corresponding aa position 741 of SEQ ID NO:191; a silent T/C polymorphism (denoted by “y” in the Sequence Listing) at nt position 2319 of SEQ ID NO:190, both of which result in a serine residue at corresponding aa position 773 of SEQ ID NO:191; a G/T polymorphism (denoted by “k” in the Sequence Listing) at nt position 2350 of SEQ ID NO:190, which can result in a glycine or cysteine residue at corresponding aa position 784 of SEQ ID NO:191; a silent A/G polymorphism (denoted by “r” in the Sequence Listing) at nt position 2161 of SEQ ID NO:190, both of which result in a proline residue at corresponding aa position 887 of SEQ ID NO:191; an A/C polymorphism (denoted by “m” in the Sequence Listing) at nt position 2765 of SEQ ID NO:190, which can result in an aspartate or alanine residue at corresponding aa position 922 of SEQ ID NO:191; a C/T polymorphism (denoted by “y” in the Sequence Listing) at nt position 2768 of SEQ ID NO:190, which can result in a leucine or proline residue at corresponding aa position 923 of SEQ ID NO:191; an A/T polymorphism (denoted by “w” in the Sequence Listing) at nt position 2773 of SEQ ID NO:190, which can result in a serine or cysteine residue at corresponding aa position 925 of SEQ ID NO:191; a C/G polymorphism (denoted by “s” in the Sequence Listing) at nt position 2186 of SEQ ID NOS:193 and 209, nt position 2168 of SEQ ID NOS:195 and 211, nt position 1868 of SEQ ID NOS:197 and 213, nt position 1829 of SEQ ID NOS:199 and 215, nt position 2447 of SEQ ID NOS:217 and 225, nt position 2429 of SEQ ID NOS:219 and 227, nt position 2129 of SEQ ID NOS:221 and 229, and nt position 2090 of SEQ ID NOS:223 and 231, which can result in a glycine or alanine residue at corresponding aa position 729 of SEQ ID NOS:194 and 210, aa position 723 of SEQ ID NOS:196 and 212, aa position 623 of SEQ ID NOS:198 and 214, aa position 610 of SEQ ID NOS:200 and 216, aa position 816 of SEQ ID NOS:218 and 226, aa position 810 of SEQ ID NOS:220 and 228, aa position 710 of SEQ ID NOS:222 and 230, and aa position 697 of SEQ ID NOS:224 and 232, respectively; a C/G polymorphism (denoted by “s” in the Sequence Listing) at nt position 1901 of SEQ ID NO:201, nt position 1883 of SEQ ID NO:203, nt position 1583 of SEQ ID NO:205, and nt position 1544 of SEQ ID NO:207, which can result in an arginine or threonine residue at corresponding aa position 634 of SEQ ID NO:202, aa position 628 of SEQ ID NO:204, aa position 528 of SEQ ID NO:206, and aa position 515 of SEQ ID NO:208, respectively; a silent C/T polymorphism (denoted by “y” in the Sequence Listing) at nt position 1857 of SEQ ID NOS:193 and 209, nt position 1839 of SEQ ID NOS:195 and 211, nt position 1539 of SEQ ID NOS:197 and 213, nt position 1500 of SEQ ID NOS:199 and 215, nt position 2118 of SEQ ID NOS:217 and 225, nt position 2100 of SEQ ID NOS:219 and 227, nt position 1800 of SEQ ID NOS:221 and 229, and nt position 1761 of SEQ ID NOS:223 and 231, both of which result in an aspartate residue at corresponding aa position 619 of SEQ ID NOS:194 and 210, aa position 613 of SEQ ID NOS:196 and 212, aa position 513 of SEQ ID NOS:198 and 214, aa position 500 of SEQ ID NOS:200 and 216, aa position 706 of SEQ ID NOS:218 and 226, aa position 700 of SEQ ID NOS:220 and 228, aa position 600 of SEQ ID NOS:222 and 230, and aa position 587 of SEQ ID NOS:224 and 232, respectively; a silent A/G polymorphism (denoted by “r” in the Sequence Listing) at nt position 1917 of SEQ ID NO:201, nt position 1899 of SEQ ID NO:203, nt position 1599 of SEQ ID NO:205, and nt position 1560 of SEQ ID NO:207, both of which result in a glutamate residue at corresponding aa position 639 of SEQ ID NO:202, aa position 633 of SEQ ID NO:204, aa position 533 of SEQ ID NO:206, and aa position 520 of SEQ ID NO:208, respectively; a C/G polymorphism at nt position 5218 of SEQ ID NOS:233 and 235, which can result in a leucine or valine residue at corresponding aa position 1740 of SEQ ID NOS:234 and 236; a C/G polymorphism at nt position 6065 of SEQ ID NO:233, which can result in an alanine or glycine residue at corresponding aa position 2022 of SEQ ID NO:234; a G/T polymorphism at nt position 111 of SEQ ID NO:237, which can result in a glutamine or histidine residue at corresponding aa position 37 of SEQ ID NO:238; a GC/CG polymorphism at nt positions 9 and 10 of SEQ ID NO:245, which can result in a proline-proline or proline-arginine dyad at corresponding aa positions 3 and 4 of SEQ ID NO:246; a C/T polymorphism at nt position 605 of SEQ ID NO:245, which can result in a serine or phenylalanine residue at corresponding aa position 202 of SEQ ID NO:246; a C/T polymorphism at nt position 637 of SEQ ID NO:245, which can result in a leucine or phenylalanine residue at corresponding aa position 213 of SEQ ID NO:246; a T/C polymorphism at nt position 1019 of SEQ ID NO:245, which can result in valine or alanine residue at corresponding aa position 340 of SEQ ID NO:246; a G/A polymorphism at nt position 1265 of SEQ ID NO:245, which can result in a serine or asparagine residue at corresponding aa position 422 of SEQ ID NO:246; a G/C polymorphism at nt position 1276 of SEQ ID NO:245, which can result in a glycine or arginine residue at corresponding aa position 426 of SEQ ID NO:246; a G/A polymorphism at nt position 1280 of SEQ ID NO:245, which can result in an arginine or glutamine residue at corresponding aa position 427 of SEQ ID NO:246; a C/A polymorphism at nt position 1709 of SEQ ID NO:245, which can result in an alanine or aspartate residue at corresponding aa position 570 of SEQ ID NO:246; a G/C polymorphism at nt position 1879 of SEQ ID NO:245, which can result in an alanine or proline residue at corresponding aa position 627 of SEQ ID NO:246; a silent A/G polymorphism at nt position 366 of SEQ ID NOS:248, 250, 252, and 254, both of which result in a glycine residue at corresponding aa position 122 of SEQ ID NOS:249, 251, 253, and 255; a G/T polymorphism at nt position 867 of SEQ ID NOS:248, 250, 252 (denoted by “k” in the Sequence Listing), and 254, which can result in a glutamine or histidine residue at corresponding aa position 289 of SEQ ID NOS:249, 251, 253, and 255; a C/G polymorphism at nt position 3686 of SEQ ID NOS:248, 252 (denoted by “s” in the Sequence Listing), and 254, and nt position 1826 of SEQ ID NO:256, which can result in a serine or tryptophan residue at corresponding aa position 1229 of SEQ ID NOS:249, 253, and 255, and aa position 609 of SEQ ID NO:257; a silent C/T polymorphism at nt position 3876 of SEQ ID NOS:248, 252, and 254, and nt position 2016 of SEQ ID NO:256, both of which result in a threonine residue at corresponding aa position 1292 of SEQ ID NOS:249, 253, and 255, and aa position 672 of SEQ ID NO:257; a silent A/G polymorphism at nt position 4800 of SEQ ID NOS:248, 252, and 254, and nt position 2940 of SEQ ID NO:256, both of which result in a glycine residue at corresponding aa position 1600 of SEQ ID NOS:249, 253, and 255, and aa position 980 of SEQ ID NO:257; a C/G polymorphism at nt position 5024 of SEQ ID NOS:248, 252, and 254, and nt position 3164 of SEQ ID NO:256, which can result in an alanine or glycine residue at corresponding aa position 1675 of SEQ ID NOS:249, 253, and 255, and aa position 1055 of SEQ ID NO:257; a silent T/G polymorphism at nt position 5091 of SEQ ID NOS:248, 252, and 254, and nt position 3231 of SEQ ID NO:256, both of which result in a glycine residue at corresponding aa position 1697 of SEQ ID NOS:249, 253, and 255, and aa position 1077 of SEQ ID NO:257; a silent C/T polymorphism at nt position 5896 of SEQ ID NOS:248, 252, and 254, and nt position 4036 of SEQ ID NO:256, both of which result in a leucine residue at corresponding aa position 1966 of SEQ ID NOS:249, 253, and 255, and aa position 1346 of SEQ ID NO:257; a silent C/T polymorphism at nt position 6777 of SEQ ID NOS:248 and 252, both of which result in a phenylalanine residue at corresponding aa position 2259 of SEQ ID NOS:249 and 253; a G/C polymorphism at nt position 427 of SEQ ID NO:258, which can result in an aspartate or histidine residue at corresponding aa position 144 of SEQ ID NO:259; a G/T polymorphism at nt position 79 of SEQ ID NO:266, which can result in an alanine or threonine residue at corresponding aa position 27 of SEQ ID NO:267; an A/G polymorphism at nt position 647 of SEQ ID NO:266, which can result in an aspartate or glycine residue at corresponding aa position 216 of SEQ ID NO:267; a silent G/A polymorphism at nt position 660 of SEQ ID NO:266, both of which result in a lysine residue at corresponding aa position 220 of SEQ ID NO:267; a silent C/T polymorphism at nt position 873 of SEQ ID NO:266, both of which result in a histidine residue at corresponding aa position 291 of SEQ ID NO:267; a G/C polymorphism at nt position 427 of SEQ ID NO:268, which can result in a glutamate or glutamine residue at corresponding aa position 143 of SEQ ID NO:269; a silent C/G polymorphism at nt position 483 of SEQ ID NO:268, both of which result in a valine residue at corresponding aa position 161 of SEQ ID NO:269; an A/G polymorphism at nt position 350 of SEQ ID NOS:272 or 274, which can result in an aspartate or glycine residue at corresponding aa position 117 of SEQ ID NOS:273 or 275; and a T/A polymorphism at nt position 1463 of SEQ ID NO:272, which can result in a valine or glutamate residue at corresponding aa position 488 of SEQ ID NO:273. The present invention contemplates sequences comprising any and all combinations and permutations of the above polymorphisms. Many of these polymorphisms are coding single nucleotide polymorphisms, and as such they are particularly useful in forensic analysis.

The genes encoding the described NHPs are apparently encoded on: human chromosome 16 (SEQ ID NOS:160-165); human chromosome 6, see GenBank Accession Number AL138876 (SEQ ID NOS:166-170); human chromosome 13, see GenBank Accession Number AL139082 (SEQ ID NOS:171-175); human chromosome 3, see GenBank Accession Number AC027483 (SEQ ID NOS:180-184); human chromosome 17, see GenBank Accession Number AC010761 (SEQ ID NOS:185-186); human chromosome 3, see GenBank Accession Number AC068979 (SEQ ID NOS:187-189); human chromosome 1, see GenBank Accession Number AL133380 (SEQ ID NOS:190-192); human chromosome 1 (SEQ ID NOS:190-192); human chromosome 6 (SEQ ID NOS:193-232); human chromosome 12 (SEQ ID NOS:233-236); human chromosome 3 and/or 2, see GenBank Accession Number AC084188 (SEQ ID NOS:237-240); a contiguous “exon” on human chromosome 9, see GenBank Accession Number AL353746 (SEQ ID NOS:241-244); human chromosome 11, see GenBank Accession Number AC018798 (SEQ ID NOS:245-247); human chromosome 6, see GenBank Accession Number AC010862 (SEQ ID NO:248-257); human chromosome 2, see GenBank Accession Number AC067825 (SEQ ID NOS:258 and 259); human chromosome 19, see GenBank Accession Number AC020922 (SEQ ID NOS:260-265); the human genomic locus defined in part by GenBank Accession Number AC023194 (SEQ ID NOS:266 and 267); human chromosome 9, see GenBank Accession Number AL161726 (SEQ ID NOS:268-271); human chromosome 3, see GenBank Accession Number AC010210 (SEQ ID NOS:272-276); and human chromosome 19, see GenBank Accession Number AC008735 (SEQ ID NOS:277-279). Accordingly, the described sequences are also useful for mapping and identifying the coding regions of the human genome, and for defining exon splice junctions.

Given the physiological importance of protein kinases, they have been subject to intense scrutiny, as exemplified and discussed in U.S. Pat. Nos. 5,532,151, 5,591,618, 5,756,289, 5,817,479, 6,001,593, and 6,340,583, which describe polynucleotides encoding similar kinase proteins, as well as a variety of uses and applications that are germane to the described NHPs.

NHP gene products can also be expressed in transgenic animals. Animals of any species, including, but not limited to, worms, mice, rats, rabbits, guinea pigs, pigs, micro-pigs, birds, goats, and non-human primates, e.g., baboons, monkeys, and chimpanzees, may be used to generate NHP transgenic animals.

Any technique known in the art may be used to introduce a NHP transgene into animals to produce the founder lines of transgenic animals. Such techniques include, but are not limited to, pronuclear microinjection (U.S. Pat. No. 4,873,191); retrovirus-mediated gene transfer into germ lines (Van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); and sperm-mediated gene transfer (Lavitrano et al., Cell 57:717-723, 1989); etc. For a review of such techniques, see, e.g., Gordon, Intl. Rev. Cytol. 115:171-229, 1989.

The present invention provides for transgenic animals that carry a NHP transgene in all their cells, as well as animals that carry a transgene in some, but not all their cells, i.e., mosaic animals or somatic cell transgenic animals. A transgene may be integrated as a single transgene, or in concatamers, e.g., head-to-head tandems or head-to-tail tandems. A transgene may also be selectively introduced into and activated in a particular cell-type by following, for example, the teaching of Lasko et al., Proc. Natl. Acad. Sci. USA 89:6232-6236, 1992. The regulatory sequences required for such a cell-type specific activation will depend upon the particular cell-type of interest, and will be apparent to those of skill in the art.

When it is desired that a NHP transgene be integrated into the chromosomal site of the endogenous NHP gene, gene targeting is preferred. Briefly, when such a technique is to be utilized, vectors containing some nucleotide sequences homologous to the endogenous NHP gene are designed for the purpose of integrating, via homologous recombination with chromosomal sequences, into and disrupting the function of the nucleotide sequence of the endogenous NHP gene (i.e., “knockout” animals).

The transgene can also be selectively introduced into a particular cell-type, thus inactivating the endogenous NHP gene in only that cell-type, by following, for example, the teaching of Gu et al., Science 265:103-106, 1994. The regulatory sequences required for such a cell-type specific inactivation will depend upon the particular cell-type of interest, and will be apparent to those of skill in the art.

Once transgenic animals have been generated, the expression of the recombinant NHP gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of tissue samples obtained from the animal, in situ hybridization analysis, and RT-PCR. Samples of NHP gene-expressing tissue may also be evaluated immunocytochemically using antibodies specific for the NHP transgene product.

The present invention also provides for “knock-in” animals. Knock-in animals are those in which a polynucleotide sequence (i.e., a gene or a cDNA) that the animal does not naturally have in its genome is inserted in such a way that it is expressed. Examples include, but are not limited to, a human gene or cDNA used to replace its murine ortholog in the mouse, a murine cDNA used to replace the murine gene in the mouse, and a human gene or cDNA or murine cDNA that is tagged with a reporter construct used to replace the murine ortholog or gene in the mouse. Such replacements can occur at the locus of the murine ortholog or gene, or at another specific site. Such knock-in animals are useful for the in vivo study, testing and validation of, intra alia, human drug targets, as well as for compounds that are directed at the same, and therapeutic proteins.

### 7.2 NHPS and NHP Polypeptides

NHPs, NHP polypeptides, NHP peptide fragments, mutated, truncated, or deleted forms of the NHPs, and/or NHP fusion proteins can be prepared for a variety of uses. These uses include, but are not limited to, the generation of antibodies, as reagents in diagnostic assays, for the identification of other cellular gene products related to a NHP, and as reagents in assays for screening for compounds that can be used as pharmaceutical reagents useful in the therapeutic treatment of mental, biological, or medical disorders and diseases. Given the similarity information and expression data, the described NHPs can be targeted (by drugs, oligos, antibodies, etc.) in order to treat disease, or to therapeutically augment the efficacy of therapeutic agents.

The Sequence Listing discloses the amino acid sequences encoded by the described NHP-encoding polynucleotides. The NHPs display initiator methionines that are present in DNA sequence contexts consistent with eucaryotic translation initiation sites. The NHPs do not display signal-like sequences, which indicates that they may not be membrane associated, and are possibly cytoplasmic or nuclear proteins, although they may also be secreted proteins.

The NHP amino acid sequences of the invention include the amino acid sequences presented in the Sequence Listing, as well as analogues and derivatives thereof. Further, corresponding NHP homologues from other species are encompassed by the invention. In fact, any NHP protein encoded by the NHP nucleotide sequences described herein are within the scope of the invention, as are any novel polynucleotide sequences encoding all or any novel portion of an amino acid sequence presented in the Sequence Listing. The degenerate nature of the genetic code is well-known, and, accordingly, each amino acid presented in the Sequence Listing is generically representative of the well-known nucleic acid “triplet” codon, or in many cases codons, that can encode the amino acid. As such, as contemplated herein, the amino acid sequences presented in the Sequence Listing, when taken together with the genetic code (see, for example, “Molecular Cell Biology”, Table 4-1 at page 109 (Darnell et al., eds., Scientific American Books, New York, N.Y., 1986)), are generically representative of all the various permutations and combinations of nucleic acid sequences that can encode such amino acid sequences.

The invention also encompasses proteins that are functionally equivalent to the NHPs encoded by the presently described nucleotide sequences, as judged by any of a number of criteria, including, but not limited to, the ability to bind and modify a NHP substrate, the ability to effect an identical or complementary downstream pathway, or a change in cellular metabolism (e.g., proteolytic activity, ion flux, phosphorylation, etc.). Such functionally equivalent NHP proteins include, but are not limited to, additions or substitutions of amino acid residues within the amino acid sequence encoded by the NHP nucleotide sequences described herein, but that result in a silent change, thus producing a functionally equivalent expression product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

A variety of host-expression vector systems can be used to express the NHP nucleotide sequences of the invention. Where the NHP peptide or polypeptide can exist, or has been engineered to exist, as a soluble or secreted molecule, the soluble NHP peptide or polypeptide can be recovered from the culture media. Such expression systems also encompass engineered host cells that express a NHP, or functional equivalent, in situ. Purification or enrichment of a NHP from such expression systems can be accomplished using appropriate detergents and lipid micelles and methods well-known to those skilled in the art. However, such engineered host cells themselves may be used in situations where it is important not only to retain the structural and functional characteristics of a NHP, but to assess biological activity, e.g., in certain drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing NHP nucleotide sequences; yeast (e.g., Saccharomyces, Pichia) transformed with recombinant yeast expression vectors containing NHP nucleotide sequences; insect cell systems infected with recombinant virus expression vectors (e.g., baculovirus) containing NHP nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing NHP nucleotide sequences; or mammalian cell systems (e.g., COS, CHO, BHK, 293, 3T3) harboring recombinant expression constructs containing NHP nucleotide sequences and promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the NHP product being expressed. For example, when a large quantity of such a protein is to be produced for the generation of pharmaceutical compositions of or containing a NHP, or for raising antibodies to a NHP, vectors that direct the expression of high levels of fusion protein products that are readily purified may be desirable. Such vectors include, but are not limited to, the E. coli expression vector pUR278 (Ruther and Muller-Hill, EMBO J. 2:1791-1794, 1983), in which a NHP coding sequence may be ligated individually into the vector in-frame with the lacZ coding region so that a fusion protein is produced; pIN vectors (Inouye and Inouye, Nucl. Acids Res. 13:3101-3109, 1985; Van Heeke and Schuster, J. Biol. Chem. 264:5503-5509, 1989); and the like. PGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target expression product can be released from the GST moiety.

In an exemplary insect system, Autographa californica nuclear polyhedrosis virus (AcNPV) is used as a vector to express foreign polynucleotide sequences. The virus grows in Spodoptera frugiperda cells. A NHP coding sequence can be cloned individually into a non-essential region (for example the polyhedrin gene) of the virus and placed under control of an AcNPV promoter (for example the polyhedrin promoter). Successful insertion of a NHP coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, the NHP nucleotide sequence of interest may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a NHP product in infected hosts (see, e.g., Logan and Shenk, Proc. Natl. Acad. Sci. USA 81:3655-3659, 1984). Specific initiation signals may also be required for efficient translation of inserted NHP nucleotide sequences. These signals include the ATG initiation codon and adjacent sequences. In cases where an entire NHP gene or cDNA, including its own initiation codon and adjacent sequences, is inserted into the appropriate expression vector, no additional translational control signals may be needed. However, in cases where only a portion of a NHP coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods in Enzymol. 153:516-544, 1987).

In addition, a host cell strain may be chosen that modulates the expression of the inserted sequences, or modifies and processes the expression product in the specific fashion desired. Such modifications (e.g., glycosylation) and processing (e.g., cleavage) of protein products may be important for the function of the protein. Different host cells have characteristic and specific mechanisms for the post-translational processing and modification of proteins and expression products. Appropriate cell lines or host systems can be chosen to ensure the desired modification and processing of the foreign protein expressed. To this end, eukaryotic host cells that possess the cellular machinery for the desired processing of the primary transcript, glycosylation, and phosphorylation of the expression product may be used. Such mammalian host cells include, but are not limited to, CHO, VERO, BHK, HeLa, COS, MDCK, 293, 3T3, WI38, and in particular, human cell lines.

For long-term, high-yield production of recombinant proteins, stable expression is preferred. For example, cell lines that stably express the NHP sequences described herein can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the a product. Such engineered cell lines may be particularly useful in screening and evaluation of compounds that affect the endogenous activity of a NHP product.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Also, anti-metabolite resistance can be used for selection with the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Alternatively, any fusion protein can be readily purified by utilizing an antibody specific for the fusion protein being expressed. Another exemplary system allows for the ready purification of non-denatured fusion proteins expressed in human cell lines (Janknecht et al., Proc. Natl. Acad. Sci. USA 88:8972-8976, 1991). In this system, the sequence of interest is subcloned into a vaccinia recombination plasmid such that the sequence's open reading frame is translationally fused to an amino-terminal tag consisting of six histidine residues. Extracts from cells infected with recombinant vaccinia virus are loaded onto Ni²⁺.nitriloacetic acid-agarose columns, and histidine-tagged proteins are selectively eluted with imidazole-containing buffers.

Also encompassed by the present invention are fusion proteins that direct a NHP to a target organ and/or facilitate transport across the membrane into the cytosol. Conjugation of NHPs to antibody molecules or their Fab fragments could be used to target cells bearing a particular epitope. Attaching an appropriate signal sequence to a NHP would also transport a NHP to a desired location within the cell. Alternatively targeting of a NHP or its nucleic acid sequence might be achieved using liposome or lipid complex based delivery systems. Such technologies are described in “Liposomes: A Practical Approach” (New, R.R.C., ed., IRL Press, New York, N.Y., 1990), and in U.S. Pat. Nos. 4,594,595, 5,459,127, 5,948,767 and 6,110,490. Additionally embodied are novel protein constructs engineered in such a way that they facilitate transport of NHPs to a target site or desired organ, where they cross the cell membrane and/or the nucleus where the NHPs can exert their functional activity. This goal may be achieved by coupling of a NHP to a cytokine or other ligand that provides targeting specificity, and/or to a protein transducing domain (see, e.g., U.S. Provisional Patent Application Ser. Nos. 60/111,701 and 60/056,713), to facilitate passage across cellular membranes, and can optionally be engineered to include nuclear localization signals.

Additionally contemplated are oligopeptides that are modeled on an amino acid sequence first described in the Sequence Listing. Such NHP oligopeptides are generally between about 10 to about 100 amino acids long, or between about 16 to about 80 amino acids long, or between about 20 to about 35 amino acids long, or any variation or combination of sizes represented therein that incorporate a contiguous region of sequence first disclosed in the Sequence Listing. Such NHP oligopeptides can be of any length disclosed within the above ranges and can initiate at any amino acid position represented in the Sequence Listing.

The invention also contemplates “substantially isolated” or “substantially pure” proteins or polypeptides. By a “substantially isolated” or “substantially pure” protein or polypeptide is meant a protein or polypeptide that has been separated from at least some of those components that naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants that accompany it in its natural state. Thus, a polypeptide that is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

### 7.3 Antibodies to NHP Products

Antibodies that specifically recognize one or more epitopes of a NHP, epitopes of conserved variants of a NHP, or peptide fragments of a NHP, are also encompassed by the invention. Such antibodies include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention can be used, for example, in the detection of a NHP in a biological sample and may, therefore, be utilized as part of a diagnostic or prognostic technique whereby patients may be tested for abnormal amounts of a NHP. Such antibodies may also be utilized in conjunction with, for example, compound screening schemes for the evaluation of the effect of test compounds on expression and/or activity of a NHP expression product. Additionally, such antibodies can be used in conjunction with gene therapy to, for example, evaluate normal and/or engineered NHP-expressing cells prior to their introduction into a patient. Such antibodies may additionally be used in methods for the inhibition of abnormal NHP activity. Thus, such antibodies may be utilized as a part of treatment methods.

For the production of antibodies, various host animals may be immunized by injection with a NHP, a NHP peptide (e.g., one corresponding to a functional domain of a NHP), a truncated NHP polypeptide (a NHP in which one or more domains have been deleted), functional equivalents of a NHP or mutated variants of a NHP. Such host animals may include, but are not limited to, pigs, rabbits, mice, goats, and rats, to name but a few. Various adjuvants may be used to increase the immunological response, depending on the host species, including, but not limited to, Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and/or coupling with molecules such as keyhole limpet hemocyanin, tetanus toxoid, diphtheria toxoid, ovalbumin, cholera toxin, or fragments thereof. Polyclonal antibodies are heterogeneous populations of antibody molecules derived from the sera of the immunized animals.

Monoclonal antibodies, which are homogeneous populations of antibodies to a particular antigen, can be obtained by any technique that provides for the production of antibody molecules by continuous cell lines in culture. These include, but are not limited to, the hybridoma technique (Kohler and Milstein, Nature 256:495-497, 1975, and U.S. Pat. No. 4,376,110), the human B-cell hybridoma technique (Kosbor et al., Immunology Today 4:72, 1983, and Cole et al., Proc. Natl. Acad. Sci. USA 80:2026-2030, 1983), and the EBV-hybridoma technique (Cole et al., in “Monoclonal Antibodies and Cancer Therapy”, Vol. 27, UCLA Symposia on Molecular and Cellular Biology, New Series, pp. 77-96 (Reisfeld and Sell, eds., Alan R. Liss, Inc. N.Y., 1985)). Such antibodies may be of any immunoglobulin class, including IgG, IgM, IgE, IgA, and IgD, and any subclass thereof. The hybridomas producing the mAbs of this invention may be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,114,598, 6,075,181 and 5,877,397. Also encompassed by the present invention is the use of fully humanized monoclonal antibodies, as described in U.S. Pat. No. 6,150,584.

Alternatively, techniques described for the production of single chain antibodies (U.S. Pat. No. 4,946,778, Bird, Science 242:423-426, 1988, Huston et al., Proc. Natl. Acad. Sci. USA 85:5879-5883, 1988, and Ward et al., Nature 341:544-546, 1989) can be adapted to produce single chain antibodies against NHP expression products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: F(ab′)₂ fragments, which can be produced by pepsin digestion of an antibody molecule; and Fab fragments, which can be generated by reducing the disulfide bridges of F(ab′)₂ fragments. Alternatively, Fab expression libraries may be constructed (Huse et al., Science 246:1275-1281, 1989) to allow rapid and easy identification of monoclonal Fab fragments with the desired specificity.

Antibodies to a NHP can, in turn, be utilized to generate anti-idiotype antibodies that “mimic” a given NHP, using techniques well-known to those skilled in the art (see, e.g., Greenspan and Bona, FASEB J. 7:437-444, 1993, and Nissinoff, J. Immunol. 147:2429-2438, 1991). For example, antibodies that bind to a NHP domain and competitively inhibit the binding of a NHP to its cognate receptor/ligand can be used to generate anti-idiotypes that “mimic” the NHP and, therefore, bind, activate, or neutralize a NHP, NHP receptor, or NHP ligand. Such anti-idiotypic antibodies, or Fab fragments of such anti-idiotypes, can be used in therapeutic regimens involving a NHP-mediated pathway.

Additionally given the high degree of relatedness of mammalian NHPs, NHP knock-out mice (having never seen a NHP, and thus never been tolerized to a NHP) have a unique utility, as they can be advantageously applied to the generation of antibodies against the disclosed mammalian NHPs (i.e., a NHP will be immunogenic in NHP knock-out animals).

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety.

1. An isolated nucleic acid molecule that encodes the amino acid sequence of SEQ ID NO:2, 4, 6, 8, 10, 12, 15, 17, 19, 22, 25, 27, 29, 31, 33, 35, 37, 39, 41, 43, 45, 47, 49, 51, 53, 55, 57, 59, 61, 63, 65, 67, 69, 71, 75, 77, 80, 83, 85, 87, 89, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 161, 163, 165, 167, 169, 172, 174, 177, 179, 181, 183, 186, 188, 191, 194, 196, 198, 200, 202, 204, 206, 208, 210, 212, 214, 216, 218, 220, 222, 224, 226, 228, 230, 232, 234, 236, 238, 240, 242, 244, 246, 249, 251, 253, 255, 257, 259, 261, 263, 265, 267, 269, 271, 273, 275, or 278.

2. The isolated nucleic acid molecule of claim 1, wherein said nucleic acid molecule comprises the nucleotide sequence of SEQ ID NO:1, 3, 5, 7, 9, 11, 14, 16, 18, 21, 24, 26, 28, 30, 32, 34, 36, 38, 40, 42, 44, 46, 48, 50, 52, 54, 56, 58, 60, 62, 64, 66, 68, 70, 74, 76, 79, 82, 84, 86, 88, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 160, 162, 164, 166, 168, 171, 173, 176, 178, 180, 182, 185, 187, 190, 193, 195, 197, 199, 201, 203, 205, 207, 209, 211, 213, 215, 217, 219, 221, 223, 225, 227, 229, 231, 233, 235, 237, 239, 241, 243, 245, 248, 250, 252, 254, 256, 258, 260, 262, 264, 266, 268, 270, 272, 274, or 277.

3. An expression vector comprising the isolated nucleic acid molecule of claim 1.

4. (canceled)

5. An isolated polypeptide comprising the amino acid sequence of SEQ ID NO:2, 4, 6, 8, 10, 12, 15, 17, 19, 22, 25, 27, 29, 31, 33, 35, 37, 39, 41, 43, 45, 47, 49, 51, 53, 55, 57, 59, 61, 63, 65, 67, 69, 71, 75, 77, 80, 83, 85, 87, 89, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 161, 163, 165, 167, 169, 172, 174, 177, 179, 181, 183, 186, 188, 191, 194, 196, 198, 200, 202, 204, 206, 208, 210, 212, 214, 216, 218, 220, 222, 224, 226, 228, 230, 253, 255, 257, 259, 261, 263, 265, 267, 269, 271, 273, 275, or 278.
